(19) United States
(12) Patent Application Publication
(10) Pub. No.: US 2025/0197920 A1
(43) Pub. Date: Jun. 19, 2025

(54) MULTI-OMIC DETERMINATION OF FERTILITY FITNESS

(71) Applicant: Virginia Tech Intellectual Properties, Inc., Blacksburg, VA (US)

(72) Inventor: Fernando H. Biase, Blacksburg, VA (US)

(21) Appl. No.: 18/982,240

(22) Filed: Dec. 16, 2024

Related U.S. Application Data
(60) Provisional application No. 63/610,485, filed on Dec. 15, 2023.

(51) Int. Cl. C12Q 1/6827; G01N 33/68

(52) U.S. Cl. CPC C12Q 1/6827; G01N 33/689; C12Q 2600/124; C12Q 2600/156; G01N 2333/4703; G01N 2333/705; G01N 2333/90245; G01N 2800/367

(57) ABSTRACT

Described in certain example embodiments herein are compositions and methods to determine fertility fitness in mammals.

### CROSS-REFERENCE TO RELATED APPLICATIONS

This application claims the benefit of and priority to U.S. Provisional Patent Application No. 63/610,485, filed on Dec. 15, 2023, the contents of which is incorporated by reference herein in its entirety.

### STATEMENT REGARDING FEDERALLY SPONSORED RESEARCH

This invention was made with government support under Grant No. 2020-67015-31616 awarded by the USDA National Institute of Food and Agriculture. The government has certain rights in the invention.

### TECHNICAL FIELD

The subject matter disclosed herein is generally directed to compositions and methods for identifying fertility status.

### BACKGROUND

Fertility issues plague both human and animals. Infertility or subfertility is a barrier to sustainable animal production, including in heifers. The development of animals that do not produce offspring within an optimum window of time can result in significant financial losses due to the input costs to raise these animals to reproductive age. Currently there is no viable approach to determining which animals are infertile or subfertile prior to detecting a pregnancy. As such, there exists an immediate need for compositions, methods, and/or techniques for identifying subfertile or infertile animals, particularly prior to any pregnancy.

Citation or identification of any document in this application is not an admission that such a document is available as prior art to the present invention.

### SUMMARY

Described in certain aspects herein are methods of assessing fertility fitness in a female mammal, the method including:
in one or more samples collected from the female mammal
(a) detecting one or more single nucleotide polymorphisms (SNPs) set forth in
(i) Supplementary Information 3;
(ii) BovineHD500034888, BovineHD500034891, BovineHD500034893, BovineHD500034894, BovineHD500034895, BovineHD500034896, BovineHD500034897, BovineHD500034898, BovineHD500034899, BovineHD1200026258, BovineHD1200026258, BovineHD2700000503, BovineHD2500009748, BovineHD2600007565, BovineHD2600007566, BovineHD1600016722, BovineHD1300006153, BovineHD2300004608, BovineHD0500034892, BovineHD0300007001, BovineHD1200026258, BovineHD2700000503, BovineHD1600016722, BovineHD0500034794, BovineHD0100008466, BovineHD2300004608, BovineHD2100009580, BovineHD2100009629, BovineHD2600011917, BovineHD0500035192, BovineHD0500035196; and/or
(iii) BovineHD500034888, BovineHD500034891, BovineHD500034893, BovineHD500034894, BovineHD500034895, BovineHD500034896, BovineHD500034897, BovineHD500034898, BovineHD500034899, BovineHD1200026258;
(b) determining an amount of a transcript of one or more genes set forth in
(iv) Supplementary Information 4;
(v) ENSBTAG00000021346, ENSBTAG00000047139, ENSBTAG0000003279, ENSBTAG0000015041, ENSBTAG0000007389, ENSBTAG0000000810, ENSBTAG0000040199, ENSBTAG0000002972, ENSBTAG0000004278, ENSBTAG0000019300, ENSBTAG00000012263, ENSBTAG00000018339, ENSBTAG00000007566, ENSBTAG00000015061, ENSBTAG00000054926, ENSBTAG00000018655, ENSBTAG00000021346, ENSBTAG00000040065, ENSBTAG00000009479, ENSBTAG0000005211, ENSBTAG00000018339, ENSBTAG00000054926, ENSBTAG00000040065, ENSBTAG00000009479, ENSBTAG00000047139, ENSBTAG00000003279, ENSBTAG00000015041, ENSBTAG00000007398, ENSBTAG00000000810, ENSBTAG00000040199;
(vi) ENSBTAG00000012263, ENSBTAG00000018339, ENSBTAG00000007566, ENSBTAG00000015061, ENSBTAG00000054926, ENSBTAG00000018655, ENSBTAG00000021346, ENSBTAG00000040065, ENSBTAG00000009479, ENSBTAG0000005211; and/or from
(vii) one or more genes selected from the group consisting of ArfGAP with SH3 domain, ankyrin repeat and PH domain 3 (ASAP3), ATP synthase membrane subunit c locus 1 (ATP5MC1), Centrosomal protein 170 (CEP170), Myeloid derived growth factor (MYDGF), Coiled-coil domain containing 34 (CCDC34), RAD51 associated protein 1 (RAD51AP1), and Ubiquinol-cytochrome c reductase complex III subunit VII (UQCRQ);
(c) determining an amount of one or more proteins set forth in
(viii) Table 2;
(ix) P00744; P02768, Q2HJF0, Q95121, A5D798, F1MZ96, Q2KIX7, G1K122, F6QND5, Q5E9E3, P19034, P02768, F1N102, F1MZ96, G3X6N3, Q2KIX7, G5E5V0, G3MY71, G1K122, Q5EA67, P19034, F1MYX5, P00744, AOA6B9SCM2, P02768, Q2HJF0, F1N102, Q95121, P80109, A5D798;
(x) P19034, F1MYX5, P00744, AOA6B9SCM2, P02768, Q2HJF0, F1N102, Q95121, P80109, A5D798; and/or
(xi) from one or more proteins selected from Apolipoprotein C-II (APOC2), Lymphocyte cytosolic protein 1 (LCP1), Vitamin K-dependent protein Z (PROZ), Albumin (ALB), Serotransferrin-like (LOC525947), Complement component C8 beta chain (C8B), Pigment epithelium-derived factor (SERPINF1), Phosphatidylinositol-glycan-specific phospholipase D (GPLD1), Alpha-ketoglutarate-dependent dioxygenase FTO (FTO); or
(e) from one or more metabolites, optionally wherein the metabolite is 2-Dehydro-D-gluconate; or
(d) any combination of (a)-(d).

Described in certain aspects herein are methods of assessing fertility fitness in a female mammal, the method including:
in one or more samples collected from the female mammal
(a) detecting the presence one or more single nucleotide polymorphisms (SNPs) selected from rs110918927, chr12: 85648422 and rs109366560, chr11:37666527;
(b) detecting and/or quantifying an amount of a transcript of adipocyte plasma membrane associated protein (APMAP), dynein axonemal intermediate chain 7 (DNAI7), or both;
(c) detecting and/or quantifying an amount of Alpha-ketoglutarate-dependent dioxygenase FTO (FTO);
(d) detecting and/or quantifying an amount of 2-Dehydro-D-gluconate; or
(e) any combination of (a)-(e).

In some aspects, detecting and/or quantifying (a), (b), (c), (d) or any combination thereof determines that the female mammal is fertile or subfertile.

In some aspects, the female mammal is fertile when the female mammal is (a) homozygous for allele A at rs110918927, chr12: 85648422, (b) is homozygous for allele G at rs109366560, chr11:37666527 (c) has a greater amount of transcript from APMAP as compared to a suitable control, (d) has a lesser amount of transcript of DNAI7 than a suitable control, (e) has a greater amount of FTO, (f) has a greater amount of 2-Dehydro-D-gluconate than a suitable control; or (g) any combination of (a)-(f).

In some aspects, the female mammal is subfertile when the female mammal (a) has at least one G allele at rs110918927, chr12: 85648422, (b) is homozygous for allele G at rs109366560, chr11:37666527, (c) has a lesser amount of transcript from APMAP as compared to a suitable control, (d) has a greater amount of transcript of DNAI7 than a suitable control, (e) has a lesser amount of FTO as compared to a suitable control, (f) has a greater amount of 2-Dehydro-D-gluconate than a suitable control; or (g) any combination of (a)-(f).

In some aspects, the female mammal is subfertile when the female mammal (a) has one A allele and one G allele or is homozygous for the G allele at rs110918927, chr12: 85648422, (b) is homozygous for allele G at rs109366560, chr11:37666527, (c) has a lesser amount of transcript from APMAP as compared to a suitable control, (d) has a greater amount of transcript of DNAI7 than a suitable control, (e) has a lesser amount of FTO as compared to a suitable control, (f) has a greater amount of 2-Dehydro-D-gluconate than a suitable control; or (g) any combination of (a)-(f).

In some aspects, the female mammal is subfertile when the female mammal has more 2-Dehydro-D-gluconate than a suitable control, optionally wherein the suitable control is a fertile female mammal.

In some aspects, the female mammal is a non-human animal.

In some aspects, the female mammal is a bovine, equine, ovine, porcine, canine, or feline.

In some aspects, the female mammal is a human.

In some aspects, the female mammal is pre-pubertal, is pubertal, or is sexually mature.

In some aspects, the one or more samples comprise a bodily fluid.

In some aspects, the one or more samples comprises blood or component thereof.

In some aspects, the one or more samples comprises plasma, buffy coat, or both.

In some aspects, the one or more samples comprises cells.

In some aspects, the cells are white blood cells.

In some aspects, the one or more samples used for detecting one or more single nucleotide polymorphisms (SNPs) comprises cells.

In some aspects, the one or more samples used for detecting and/or quantifying an amount of a transcript comprises cells.

In some aspects, the one or more samples comprises blood buffy coat.

In some aspects, the cells include white blood cells.

In some aspects, the one or more samples used for detecting and/or quantifying an amount of a protein comprises blood or a component thereof.

In some aspects, the one or more samples used for detecting and/or quantifying an amount of a protein comprises blood plasma.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1E—Overview of data produced. (FIG. 1A) Breeds and classification used in this study, including sample size. (FIG. 1B) Schematics of the data produced, and analysis undertaken. Principal component analysis of the genome-wide single nucleotide polymorphisms (FIG. 1C), transcriptome (FIG. 1D) and (FIG. 1E) proteome data. GWAS: genome-wide association analysis, DGE: differential gene expression, DPA: differential protein abundance.

FIG. 2A-2B—Genome-wide association analysis of fertility in beef and dairy heifers. (FIG. 2A) Manhattan plot with the distribution of SNPs across their genome and their P values from Fisher's exact association test. (FIG. 2B) Genetic frequencies of the two SNPs that are putatively linked to fertility in heifers.

FIG. 3A-3B—Differential transcript and protein abundance associated with fertility. (FIG. 3A) Transcript abundance. (FIG. 3B) Protein abundance. In each plot, within each breed, shapes indicate the same animal.

FIG. 4A-4D—Multi-omics analysis of heifer fertility. (FIG. 4A) Percentage of variance explained by each factor within each data modality. (FIG. 4B) Relative separation of samples based on their phenotype for each factor plotted. (FIG. 4C) The relative weight of importance of the top ten features (SNP identifiers are from the array annotation, gene identifiers are from Ensembl, protein identifiers are from UniProt) in each data modality and factor plotted. (FIG. 4D) Sample clustering using all three data modes.

FIG. 5A-5B—Metabolites in the plasma of heifers. (FIG. 5A) Forty most abundant metabolites detected in the plasma of heifers at breeding (mean±standard deviation). (FIG. 5B) Differential abundance of 2-dehydro-d-gluconate based on the reproductive outcome of Angus and Holstein heifers. Dots represent the data points; diamond represent the mean and error bars represent one standard deviation.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, ‘less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, ‘greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et al., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a” “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g., a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Infertility or subfertility is a critical barrier to sustainable cattle production, including in heifers. The development of heifers that do not produce a calf within an optimum window of time is a critical factor for the profitability and sustainability of the cattle industry. In parallel, heifers are an excellent biomedical model for understanding the underlying etiology of infertility because well-nourished heifers can still be infertile, mostly because of inherent physiological and genetic causes.

Exemplary embodiments disclosed herein set forth multi-omic determination of fertility status (fertile or subfertile/infertile) in a female mammal. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Methods of Determining Fertility Fitness

The present disclosure provides biomarkers (e.g., single nucleotide polymorphisms (SNPs), phenotype (transcripts, proteins) for the determination of fertility fitness of a female mammal (e.g., fertile vs. infertile or subferitle female mammals). Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature SNPs, genes, and/or signature gene products as described herein. Biomarkers of the present disclosure are useful in methods of determining fertility fitness of a female mammal.

For example, distinct reference values may represent the probability or likelihood that a female mammal subject is fertile or infertile/subfertile. In another example, distinct reference values may represent predictions of differing degrees of probability of being fertile or infertile/subfertile. Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular condition (i.e., for whom said condition holds true). In some embodiments, the condition is fertile. In some embodiments, the condition is infertile/subfertile. Such population may comprise without limitation 2 or more, 10 or more, 100 or more, or even several hundred or more individuals. It will be appreciated that such reference values can be considered suitable controls or threshold values that can be used in the context of a method of the present disclosure.

A test value (i.e., a value obtained from a test sample) may deviate from a reference value or threshold value. In some embodiments, a test value that indicates the fertile condition deviates from a reference value or threshold value. In some of these embodiments, the reference value or threshold value was determined using known fertile female samples. In some of these embodiments, the reference value or threshold value was determined using known infertile/subfertile female samples. In some embodiments, a test value that indicates the subfertile condition deviates from a reference value or threshold value.

A “deviation” of a first value from a second value can generally encompass any direction (e.g., increase: first value>second value; or decrease: first value<second value) and any extent of alteration.

For example, a deviation can encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation can encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation can refer to a statistically significant observed alteration. For example, a deviation can refer to an observed alteration which falls outside of error margins of reference values in a given population (as expressed, for example, by standard deviation or standard error, or by a predetermined multiple thereof, e.g., ±1×SD or ±2×SD or ±3×SD, or 1×SE or ±2×SE or ±3×SE). Deviation can also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation can be concluded if an observed alteration is beyond a given threshold or cut-off. Such threshold or cut-off can be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

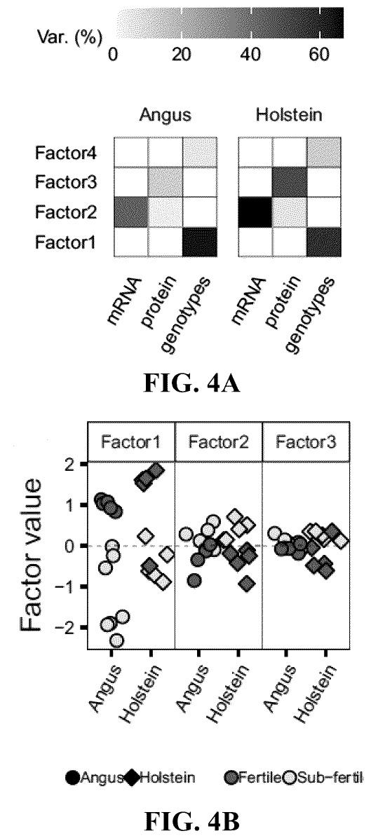

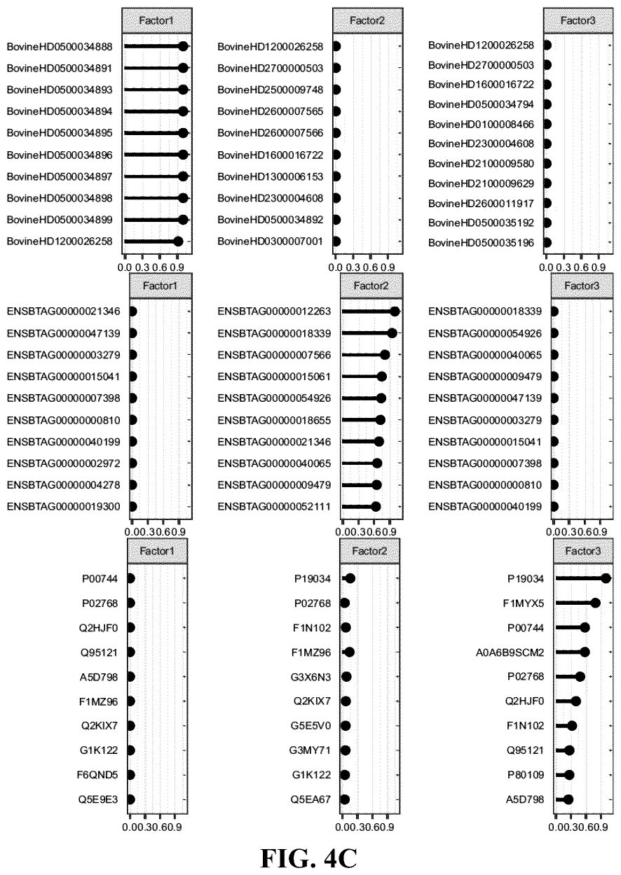

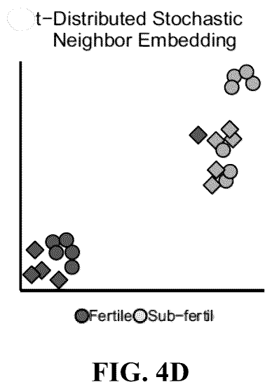

In some embodiments, a method of assessing fertility fitness in a female mammal, the method including in one or more samples collected from the female mammal (a) detecting one or more single nucleotide polymorphisms (SNPs) set forth in (i) Supplementary Information 3, (ii) FIG. 4C (BovineHD500034888, BovineHD500034891, BovineHD500034893, BovineHD500034894, BovineHD500034895, BovineHD500034896, BovineHD500034897, BovineHD500034898, BovineHD500034899, BovineHD1200026258, BovineHD1200026258, BovineHD2700000503, BovineHD2500009748, BovineHD2600007565, BovineHD2600007566, BovineHD1600016722, BovineHD1300006153, BovineHD2300004608, BovineHD0500034892, BovineHD0300007001, BovineHD1200026258, BovineHD2700000503, BovineHD1600016722, BovineHD0500034794, BovineHD0100008466, BovineHD2300004608, BovineHD2100009580, BovineHD2100009629, BovineHD2600011917, BovineHD0500035192, BovineHD0500035196), and/or (iii) Factor 1 of FIG. 4C (BovineHD500034888, BovineHD500034891, BovineHD500034893, BovineHD500034894, BovineHD500034895, BovineHD500034896, BovineHD500034897, BovineHD500034898, BovineHD500034899, BovineHD1200026258); (b) determining an amount of a transcript of one or more genes set forth in (iv) Supplementary Information 4; (v) FIG. 4C (ENSBTAG00000021346, ENSBTAG00000047139, ENSBTAG0000003279, ENSBTAG0000015041, ENSBTAG0000007389, ENSBTAG0000000810, ENSBTAG0000040199, ENSBTAG0000002972, ENSBTAG0000004278, ENSBTAG0000019300, ENSBTAG00000012263, ENSBTAG00000018339, ENSBTAG00000007566, ENSBTAG00000015061, ENSBTAG00000054926, ENSBTAG00000018655, ENSBTAG00000021346, ENSBTAG00000040065, ENSBTAG00000009479, ENSBTAG0000005211, ENSBTAG00000018339, ENSBTAG00000054926, ENSBTAG00000040065, ENSBTAG00000009479, ENSBTAG00000047139, ENSBTAG00000003279, ENSBTAG00000015041, ENSBTAG00000007398, ENSBTAG00000000810, ENSBTAG00000040199); (vi) Factor 2 of FIG. 4C (ENSBTAG00000012263, ENSBTAG00000018339, ENSBTAG00000007566, ENSBTAG00000015061, ENSBTAG00000054926, ENSBTAG00000018655, ENSBTAG00000021346, ENSBTAG00000040065, ENSBTAG00000009479, ENSBTAG0000005211); and/or from (vii) one or more genes selected from the group consisting of ArfGAP with SH3 domain, ankyrin repeat and PH domain 3 (ASAP3), ATP synthase membrane subunit c locus 1 (ATP5MC1), Centrosomal protein 170 (CEP170), Myeloid derived growth factor (MYDGF), Coiled-coil domain containing 34 (CCDC34), RAD51 associated protein 1 (RAD51AP1), and Ubiquinol-cytochrome c reductase complex III subunit VII (UQCRQ); (c) determining an amount of one or more proteins set forth in (viii) Table 2; (ix) FIG. 4C (P00744; P02768, Q2HJF0, Q95121, A5D798, F1MZ96, Q2KIX7, G1K122, F6QND5, Q5E9E3, P19034, P02768, F1N102, F1MZ96, G3X6N3, Q2KIX7, G5E5V0, G3MY71, G1K122, Q5EA67, P19034, F1MYX5, P00744, AOA6B9SCM2, P02768, Q2HJF0, F1N102, Q95121, P80109, A5D798); (x) Factor 3 of FIG. 4C (P19034, F1MYX5, P00744, AOA6B9SCM2, P02768, Q2HJF0, F1N102, Q95121, P80109, A5D798); and/or (xi) from one or more proteins selected from Apolipoprotein C-II (APOC2), Lymphocyte cytosolic protein 1 (LCP1), Vitamin K-dependent protein Z (PROZ), Albumin (ALB), Serotransferrin-like (LOC525947), Complement component C8 beta chain (C8B), Pigment epithelium-derived factor (SERPINF1), Phosphatidylinositol-glycan-specific phospholipase D (GPLD1), Alpha-ketoglutarate-dependent dioxygenase FTO (FTO); (d) from one or more metabolites, optionally wherein the metabolite is 2-Dehydro-D-gluconate; or (e) any combination of (a)-(d). In some embodiments, detecting and/or quantifying (a), (b), (c), (d) or any combination thereof determines that the female mammal is fertile or subfertile.

In some embodiments, a method of assessing fertility fitness in a female mammal, the method including: in one or more samples collected from the female mammal (a) detecting the presence one or more single nucleotide polymorphisms (SNPs) selected from rs110918927, chr12: 85648422 and rs109366560, chr11:37666527; (b) detecting and/or quantifying an amount of a transcript of adipocyte plasma membrane associated protein (APMAP), dynein axonemal intermediate chain 7 (DNAI7), or both; (c) detecting and/or quantifying an amount of Alpha-ketoglutarate-dependent dioxygenase FTO (FTO); (d) detecting and/or quantifying an amount of 2-Dehydro-D-gluconate; or (e) any combination of (a)-(d). In some embodiments, detecting and/or quantifying (a), (b), (c), (d) or any combination thereof determines that the female mammal is fertile or subfertile.

In some embodiments, the female mammal is fertile when the female mammal (a) is homozygous for allele A at rs110918927, chr12: 85648422, (b) is homozygous for allele G at rs109366560, chr11:37666527 (c) has a greater amount of transcript from APMAP as compared to a suitable control, (d) has a lesser amount of transcript of DNAI7 than a suitable control, (e) has a greater amount of FTO as compared to a suitable control, (f) a greater amount of 2-Dehydro-D-gluconate as compared to a suitable control; or any combination of (a)-(f). In some embodiments, detecting and/or quantifying (a), (b), (c), (d), (e), (f), or any combination thereof determines that the female mammal is fertile or subfertile.

In some embodiments, the female mammal is subfertile when the female mammal (a) has at least one G allele at rs110918927, chr12: 85648422, (b) is homozygous for allele G at rs109366560, chr11:37666527, (c) has a lesser amount of transcript from APMAP as compared to a suitable control, (d) has a greater amount of transcript of DNAI7 than a suitable control, (e) has a lesser amount of FTO as compared to a suitable control, (f) a greater amount of 2-Dehydro-D-gluconate as compared to a suitable control; or any combination of (a)-(f). In some embodiments, the female mammal is subfertile when the female mammal (a) has one A allele and one G allele or is homozygous for the G allele at rs110918927, chr12: 85648422, (b) is homozygous for allele G at rs109366560, chr11:37666527 (c) has a lesser amount of transcript from APMAP as compared to a suitable control, (d) has a greater amount of transcript of DNAI7 than a suitable control, (e) has a lesser amount of FTO as compared to a suitable control, (f) has a a greater amount of 2-Dehydro-D-gluconate as compared to a suitable control; or (g) any combination of (a)-(f).

In some embodiments, the suitable control is a sample from a fertile female mammal. In some embodiments, the suitable control is a sample from an infertile/subfertile female mammal. In some embodiments, the suitable control is a reference or threshold value determined from a fertile female mammal or fertile female mammal population. In some embodiments, the suitable control is a reference or threshold value determined from an infertile/subfertile female mammal or infertile/subfertile female mammal population.

In some embodiments, where the transcript amount or protein amount is greater than a suitable control, the transcript amount or protein amount is 0.1-100 fold or more greater than the suitable control. In some embodiments, where the transcript amount or protein amount is less than a suitable control, the transcript amount or protein amount is 0.1-100 fold or more less than the suitable control.

Quantifying can be relative or absolute quantification. Methods of relative and absolute quantification of the biomarkers of the present disclosure will be appreciated by those of ordinary skill in the art. In some embodiments, detection is high throughput. In some embodiments, detection is not high throughput. In some embodiments, detection is at point of care.

Methods and techniques for detecting and/or quantifying DNA, RNA, and/or proteins are generally known in the art. Exemplary methods/techniques include, without limitation, PCR based assays (e.g., PCR, RT-PCR, qPCR, qRT-PCT, etc.), Northern blot analysis, Southern blot analysis, Western blot analysis, sequencing methods (Sanger, next generation sequencing techniques, etc.), chromatography, size exclusion methods, immunofluorescence, ELISA, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. In some embodiments, the analytic technique is high-throughput. Other methods including absorbance assays and colorimetric assays are known in the art and can be used herein. detection can include primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

### MS Methods

Biomarker detection can also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results can be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests can be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they can absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation can include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which can be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, can be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### Single Cell Sequencing

In certain embodiments, the method involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the method involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the method involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the method involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the method involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

### CRISPR-Cas Based Detection Methods

Non-specific collateral RNase activity of a Cas13 or Cas12a can be leveraged to cleave reporters upon target recognition, allowing for the design of sensitive and specific diagnostics using a Cas13 or Cas12a, including single nucleotide variants, detection based on rRNA sequences, screening for drug resistance, monitoring microbe outbreaks, genetic perturbations, and screening of environmental samples, as described, for example, in PCT/US18/054472 filed Oct. 22, 2018 at [0183]-[0327], incorporated herein by reference. Reference is made to WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and U.S. Pat. No. 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26, 2018 entitled “Novel Cas12b Enzymes and Systems”, U.S. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox D B T, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6., Science. 2018 Apr. 27; 360(6387):439-444; Gootenberg J S, et al., Nucleic acid detection with CRISPR-Cas13a/C2c2, Science. 2017 Apr. 28; 356(6336):438-442; Abudayyeh 00, et al., RNA targeting with CRISPR-Cas13, Nature. 2017 Oct. 12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65(4):618-630.e7; Abudayyeh 00, et al., C2c2 is a single-component programmable RNA-guided RNA-targeting CRISPR effector, Science. 2016 Aug. 5; 353(6299):aaf5573; Yang L, et al., Engineering and optimising deaminase fusions for genome editing. Nat Commun. 2016 Nov. 2; 7:13330, Myrvhold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, each of which is incorporated herein by reference in its entirety.

In some embodiments, female non-human mammals determined to be fertile are bred and/or maintained as breeding stock. In some embodiments, female non-human mammals determined to be fertile are removed from breeding stock.

In some embodiments, the female mammal is a non-human animal. In some embodiments, the female mammal is a bovine, equine, ovine, porcine, canine, or feline. In some embodiments, the female mammal is a human. In some embodiments, the female mammal is pre-pubertal, is pubertal, or is sexually mature.

In some embodiments, the one or more samples include a bodily fluid. In some embodiments, the one or more samples includes blood or component thereof. In some embodiments, the one or more samples includes plasma, buffy coat, or both. In some embodiments, the one or more samples includes cells. In some embodiments, the cells are white blood cells. In some embodiments, one or more samples used for detecting one or more single nucleotide polymorphisms (SNPs) includes cells. In some embodiments, wherein the one or more samples used for detecting and/or quantifying an amount of a transcript includes cells. In some embodiments, the one or more samples includes blood buffy coat. In some embodiments, wherein the cells include white blood cells. In some embodiments, the one or more samples used for detecting and/or quantifying an amount of a protein includes blood or a component thereof. In some embodiments, the one or more samples used for detecting and/or quantifying an amount of a protein includes blood plasma.

In some embodiments, the sample is 1-1,000 pL, nL, μL, or mL. In some embodiments, the sample is 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 pL, nL, μL, or mL.

### Kits

Described in example embodiments herein are kits, such as combination kits, that contain one or more compounds, compositions, formulations, devices, for performing a method described herein. As used herein, the terms “combination kit” or “kit of parts” refers to the compounds, compositions, formulations, particles, devices, and any additional components that are used to collect samples, store samples, ship samples, test samples, package, sell, market, deliver. In some embodiments, the kit comprises one or more detection reagents. Such additional components include, but are not limited to, packaging, syringes, blister packages, bottles, solutions, detection reagents, swabs, collection vials, collection tubes, labels and the like.

In some embodiments, a kit includes one or more reagents capable of or adapted for detecting and/or quantifying, in a sample, a SNP, a transcript abundance, and/or protein abundance of one or more SNPs, of one or more genes, and/or one or more proteins described elsewhere herein.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

### Introduction

The latest data from the Food and Agriculture Organization show that in 2020 more than 46% of the daily protein supply in the world was from animal-based foods (FAO-STATS). Bovine meat and milk accounted for 12.8% of the total protein supply in the world in 2020 (FAO-STATS). These numbers underscore the importance of cattle production to sustain a growing demand for protein globally¹. Infertility or subfertility is a critical barrier to sustainable cattle production², including in heifers. For example, approximately 15%³ and 5%⁴ of beef and dairy heifers, respectively, do not calve at 24 months of age. Heifers that calve at an optimum age have greater productivity and longevity in the herd^{5,6,7,8,9,10}. Therefore, identifying heifers with optimum fertility is a promising approach to improving sustainability in cattle production.

The heritability of breeding values for heifer fertility is often low for beef^{11,12,13,14,15,16,17} and dairy^{18,19,20,21,22,23} heifers, which indicate that there are multiple genetic factors impacting this complex trait beyond additive genetic effects. Another potential avenue for the understanding of infertility is the use of molecular phenotyping²⁴. The pioneering efforts focused on genome-wide association studies (GWAS) to identify genetic markers associated with heifer fertility^{4,12,25,26,27,28,29,30,31,32,33,34,35,36,37,38} but only a few seem to be reproducible across populations³⁷. More recent efforts have also focused on transcriptome^{39,40,41} and metabolome⁴² datasets characterizing these molecules in blood samples. Again, limited genes have been identified with differential transcript abundance across datasets³⁹. Much research is needed for the identification of molecular features that can help explain fertility fitness.

Altogether, approximately 5% of heifers are infertile^{4,43}, and this cohort is a great biological model for studying the genetic bases of infertility for several reasons. First, neither dairy nor beef heifers are under the challenging metabolic demand required for milk production^{44,45,46}. Second, post-partum cows need to undergo a critical period of physiological and anatomical recovery before the next breeding^{47,48,49}. Third, there are several postpartum diseases with negative consequences on reproduction success^{50,51,52}. Reproductive problems in well-managed heifers are inherent to their physiology²⁰, most of which are also under genetic control⁵³, or directly related to mutations⁵⁴ that impair female reproductive functions.

Angus and Holstein heifers have similar frequencies of infertility or subfertility^{4,43} despite the selection pressures directed at beef or dairy production, and thus have distant genetic background. Most studies involving the identification of biological features associated with fertility in heifers have used either one group of purebred or crossbreed animal^{4,12,25,26,27,28,29,30,31,32,33,34,35,36,37,38,39,40,41,42}. Here, Applicant carried out a case-control⁵⁵ experiment to test the hypothesis that differences in genetic variants, gene transcript, and protein abundance due to fertility fitness would be shared between heifers of different genetic background. Our objective was to contrast genetic variants, gene transcripts, and protein abundance between Fertile and Sub-Fertile heifers from Angus and Holstein genetic backgrounds. Applicant shows that both the independent analysis and multi-omics approach identified molecular signatures that capable of discriminating heifers of differing fertility potential, and thus with an underlying biology associated with fertility that is shared between both breeds.

### Methods

For additional methods see Appendix A to U.S. Provisional Application 63/610,485 and Marrella and Biase et al., Sci. Rep. 2023. 13(1): 12664, which are incorporated by reference herein in their entireties.

### Experimental Design

Applicant collected blood samples from purebred Angus heifers (n=12), averaging 14 months in age, at the time of their first artificial insemination (AI) service. Heifers were subjected to a 7-Day Co-Synch+CIDR estrus synchronization protocol prior to breeding. Briefly, heifers were administered an intramuscular (IM) injection of gonadotrophin-releasing hormone (GnRH, 100 g; Factrel®; Zoetis Inc.) on Day 0, followed by the insertion of a controlled internal drug release (CIDR, 1.38 g Progesterone; Eazi-Breed™ CIDR®; Zoetis Inc.). On Day 7, the CIDR was removed and an injection of prostaglandin F2 alpha (PGF2α, 25 μg; Lutalyse®; Zoetis Inc.) was delivered. Fixed-time AI was performed 54±2 h following CIDR removal alongside a second injection of GnRH.

Additionally, Applicant collected blood samples from purebred Holstein (n=10) heifers, averaging 12 months in age, at the time of the first AI service. Heifers were enrolled in a 5-Day CIDR-Synch protocol before insemination. Briefly, an IM injection of GnRH was delivered on Day 0 with the insertion of a CIDR device. The CIDR device was removed on Day 5, followed by an IM injection of PGF2a. A second injection of PGF2a was administered 24 hours later. Then, timed AI was performed with a second GnRH injection on Day 8.

Heifers were identified as Fertile (Holstein, n=5; Angus, n=5) or Sub-Fertile (Holstein, n=5; Angus, n=7) based on their pregnancy outcome, following similar criteria used previously^{39,40}. Fertile animals were identified as those who became pregnant and subsequently delivered a calf following the first insemination service. Angus heifers were categorized as Sub-Fertile after failing to achieve pregnancy following two insemination services and exposure to a bull for natural breeding. Holstein heifers were identified as Sub-Fertile after needing four or more artificial inseminations.

Heifers were synchronized with protocols that have been identified by prior research to have high success for a heifer to become pregnant to AI^{56,57}. Hence the different protocols for beef and dairy heifers. The criteria for classification were different for each group due to differences in management that are inherent to beef and dairy replacement heifers. Most importantly, each heifer had multiple opportunities to become pregnant before being classified as sub-fertile.

The heifers utilized in this study were not part of a nutritional experiment, and thus nutrition was not accounted as a variable nor was it a factor in the selection of heifers. All dairy heifers were raised with equivalent exposure to feed. Similarly, all beef heifers were raised with equivalent exposure to feed.

### Blood Sample Collection and White Blood Cell Isolation

Fifty ml of blood were drawn from each animal by venipuncture of the jugular vein using 18 mg K2 EDTA vacutainers (Becton, Dickinson, and Company). The tubes were inverted for proper mixing with the anticoagulant and then immediately placed on ice until further processing.

Applicant processed the blood samples following procedures described elsewhere^{39,40,58} within three hours of sampling⁵⁸. Tubes containing whole blood samples were centrifuged for 25 minutes (min) at 4° C. and 2000×g to separate the buffy coat. The buffy coat was then aspirated and mixed with 14 ml of red blood cell lysis buffer (1.55 M ammonium chloride, 0.12 M sodium bicarbonate, 1 mM EDTA (Cold Spring Harbor Protocols)). Then, the solution was centrifuged for 10 min at 4° C. at 800×g and the supernatant was discarded. The remaining pellet was mixed with 200 μl TRIzol™ Reagent (Invitrogen™, Thermo Fisher Scientific, Waltham, MA) in a 2 ml cryotube (Corning Inc., Corning, NY) prior to snap-freezing with liquid nitrogen. Samples were then stored at −80° C. until further processing.

### Total RNA and DNA Extraction

The buffy coat samples were thawed at room temperature in a total volume of 525 μl TRIzol™ Reagent. Then, total RNA was extracted from peripheral white blood cells using the Zymo Research Direct-zol™ DNA/RNA Miniprep kit (Zymo Research Corporation, Irvine, CA), according to the manufacturer's protocol. Next, Applicant assessed the quality of the RNA by quantifying the RNA integrity number (RIN) for each sample using the Agilent RNA 6000 Pico kit (Agilent, Santa Clara, CA) on the Agilent 2100 Bioanalyzer (Agilent, Santa Clara, CA).

### Genotyping and Data Processing

Applicant submitted 400 ng of DNA for each heifer to Neogen (Neogen Corporation, Lincoln, NE) for genotyping. The samples were genotyped using the Illumina BovineHD Beadchip (Illumina Inc., San Diego, CA) genotyping array (777K). Applicant processed the data for quality control⁵⁹ using PLINKL⁶⁰. First, Applicant removed SNPs that were preferentially called in one of the groups in the case and control. This was followed by the removal of samples with more than 10% of the genotypes missing, and removal of SNPs with a minor allelic frequency less than 1%, a missing rate greater than 10%, or deviation from the Hardy-Weinberg equilibrium (P<0.00001). Next, Applicant carried out variant pruning. Applicant considered a window size of 50 kilobases with five variants in each window at a correlation threshold of 0.2. After pruning, Applicant calculated relatedness and inbreeding coefficients using the parameter ‘--make-rel’ in PLINK (Table 1). All reported SNP coordinates are relative to btau9 assembly converted with the LiftOver tool⁶¹.

TABLE 1
Estimates of relatedness (lower triangle) and inbreeding (diagonal)

| Breed | Sample ID | Sample_1 | Sample_2 | Sample_3 | Sample_4 | Sample_5 | Sample_6 | Sample_7 | Sample_8 |
|---|---|---|---|---|---|---|---|---|---|
| Holstein | Sample_1 | 1.08614 | | | | | | | |
| Holstein | Sample_2 | 0.175852 | 1.0622 | | | | | | |
| Holstein | Sample_3 | 0.132934 | 0.0786326 | 1.07225 | | | | | |
| Holstein | Sample_4 | 0.211885 | 0.117685 | 0.287008 | 1.019 | | | | |
| Holstein | Sample_5 | 0.129678 | 0.127473 | 0.141105 | 0.10591 | 1.14286 | | | |
| Holstein | Sample_6 | 0.0898733 | 0.129848 | 0.149044 | 0.084637 | 0.268721 | 1.13966 | | |
| Holstein | Sample_7 | 0.197396 | 0.175516 | 0.146799 | 0.216424 | 0.179404 | 0.14415 | 1.03216 | |
| Holstein | Sample_8 | 0.214026 | 0.32936 | 0.100687 | 0.186373 | 0.0950101 | 0.117043 | 0.156906 | 1.02237 |
| Holstein | Sample_9 | 0.130305 | 0.0878428 | 0.32471 | 0.373388 | 0.126073 | 0.0784041 | 0.172895 | 0.108189 |
| Holstein | Sample_10 | 0.182636 | 0.269277 | 0.091545 | 0.123844 | 0.0717739 | 0.173054 | 0.144058 | 0.323249 |
| Angus | Sample_11 | −0.22036 | −0.222252 | −0.216947 | −0.239237 | −0.205892 | −0.194061 | −0.226336 | −0.229944 |
| Angus | Sample_12 | −0.214415 | −0.216803 | −0.212921 | −0.22485 | −0.187671 | −0.196102 | −0.209688 | −0.221599 |
| Angus | Sample_13 | −0.198503 | −0.199367 | −0.197146 | −0.220744 | −0.187528 | −0.19329 | −0.205576 | −0.202728 |
| Angus | Sample_14 | −0.213995 | −0.215747 | −0.21939 | −0.231431 | −0.197697 | −0.191915 | −0.215429 | −0.230278 |
| Angus | Sample_15 | −0.216705 | −0.213112 | −0.197847 | −0.224818 | −0.198714 | −0.205621 | −0.210562 | −0.211926 |
| Angus | Sample_16 | −0.212651 | −0.208834 | −0.2239 | −0.230988 | −0.199043 | −0.202886 | −0.21981 | −0.231616 |
| Angus | Sample_17 | −0.209613 | −0.210616 | −0.208132 | −0.226408 | −0.190069 | −0.204498 | −0.210912 | −0.218407 |
| Angus | Sample_18 | −0.225846 | −0.227054 | −0.222804 | −0.233677 | −0.209164 | −0.209519 | −0.227585 | −0.226917 |
| Angus | Sample_19 | −0.206436 | −0.199351 | −0.200885 | −0.223839 | −0.191914 | −0.189758 | −0.202618 | −0.211767 |
| Angus | Sample_20 | −0.225245 | −0.222195 | −0.217755 | −0.237027 | −0.21063 | −0.198929 | −0.223326 | −0.234552 |
| Angus | Sample_21 | −0.192314 | −0.204261 | −0.202353 | −0.212709 | −0.2009 | −0.189902 | −0.205304 | −0.212034 |
| Angus | Sample_22 | −0.214969 | −0.214432 | −0.205074 | −0.22065 | −0.209137 | −0.198262 | −0.208887 | −0.221685 |

| Breed | Sample ID | Sample_9 | Sample_10 | Sample_11 | Sample_12 | Sample_13 | Sample_14 | Sample_15 | Sample_16 |
|---|---|---|---|---|---|---|---|---|---|
| Holstein | Sample_1 | | | | | | | | |
| Holstein | Sample_2 | | | | | | | | |
| Holstein | Sample_3 | | | | | | | | |
| Holstein | Sample_4 | | | | | | | | |
| Holstein | Sample_5 | | | | | | | | |
| Holstein | Sample_6 | | | | | | | | |
| Holstein | Sample_7 | | | | | | | | |
| Holstein | Sample_8 | | | | | | | | |
| Holstein | Sample_9 | 1.08627 | | | | | | | |
| Holstein | Sample_10 | 0.0956321 | 1.04665 | | | | | | |
| Angus | Sample_11 | −0.226962 | −0.217662 | 0.977219 | | | | | |
| Angus | Sample_12 | −0.225013 | −0.212688 | 0.0528899 | 1.00826 | | | | |
| Angus | Sample_13 | −0.193574 | −0.196643 | 0.0543309 | 0.104217 | 1.05989 | | | |
| Angus | Sample_14 | −0.221758 | −0.214065 | 0.100656 | 0.276834 | 0.0467522 | 1.00802 | | |
| Angus | Sample_15 | −0.215619 | −0.212666 | 0.190602 | 0.0915124 | 0.0826358 | 0.0725822 | 0.987252 | |
| Angus | Sample_16 | −0.221677 | −0.209096 | 0.172326 | 0.0863896 | 0.0644944 | 0.125298 | 0.0760984 | 0.960192 |
| Angus | Sample_17 | −0.212742 | −0.206144 | 0.0802295 | 0.108972 | 0.106461 | 0.114032 | 0.0807007 | 0.054771 |
| Angus | Sample_18 | −0.213991 | −0.21562 | 0.145322 | 0.0855901 | 0.128681 | 0.0628431 | 0.0849965 | 0.128438 |
| Angus | Sample_19 | −0.205592 | −0.206668 | 0.100707 | 0.0618605 | 0.0847171 | 0.0796406 | 0.133815 | 0.0737866 |
| Angus | Sample_20 | −0.224743 | −0.218116 | 0.246251 | 0.0538049 | 0.070959 | 0.101206 | 0.126725 | 0.169895 |
| Angus | Sample_21 | −0.214643 | −0.201884 | 0.0373366 | 0.0860991 | 0.092535 | 0.0899922 | 0.0755912 | 0.165132 |
| Angus | Sample_22 | −0.207679 | −0.210778 | 0.0412866 | 0.104891 | 0.0988938 | 0.0733013 | 0.104626 | 0.083242 |

| Breed | Sample ID | Sample_17 | Sample_18 | Sample_19 | Sample_20 | Sample_21 | Sample_22 |
|---|---|---|---|---|---|---|---|
| Holstein | Sample_1 | | | | | | |
| Holstein | Sample_2 | | | | | | |
| Holstein | Sample_3 | | | | | | |
| Holstein | Sample_4 | | | | | | |
| Holstein | Sample_5 | | | | | | |
| Holstein | Sample_6 | | | | | | |
| Holstein | Sample_7 | | | | | | |
| Holstein | Sample_8 | | | | | | |
| Holstein | Sample_9 | | | | | | |
| Holstein | Sample_10 | | | | | | |
| Angus | Sample_11 | | | | | | |
| Angus | Sample_12 | | | | | | |
| Angus | Sample_13 | | | | | | |
| Angus | Sample_14 | | | | | | |
| Angus | Sample_15 | | | | | | |
| Angus | Sample_16 | | | | | | |
| Angus | Sample_17 | 1.07157 | | | | | |
| Angus | Sample_18 | 0.132202 | 1.01485 | | | | |
| Angus | Sample_19 | 0.0775906 | 0.0919451 | 1.03382 | | | |
| Angus | Sample_20 | 0.0477571 | 0.166557 | 0.0761634 | 0.997098 | | |
| Angus | Sample_21 | 0.0721961 | 0.0800696 | 0.0973602 | 0.079813 | 1.0204 | |
| Angus | Sample_22 | 0.150579 | 0.0902108 | 0.126952 | 0.0758494 | 0.139294 | 1.02192 |

### Library Preparation and Sequencing

For sequencing library construction, 900 ng of total RNA was diluted into 25 μl of nuclease-free water, and RNA quantity was confirmed using the Qubit™ RNA High Sensitivity Assay kit (Invitrogen™, Thermo Fisher Scientific, Waltham, MA) on the Qubit™ 4 Fluorometer (Invitrogen™, Thermo Fisher Scientific, Waltham, MA). Libraries were prepared for next-generation sequencing using the Illumina Stranded mRNA Prep kit (Illumina, Inc., San Diego, CA) and the IDT® for Illumina RNA UD indexes (Illumina, Inc., San Diego, CA) according to the manufacturer's instructions. Sequencing was conducted on the NovaSeq 6000 sequencing system (Illumina, Inc., San Diego, CA) using the NovaSeq 6000 SP Reagent kit v1.5 (Illumina, Inc., San Diego, CA) to produce paired-end reads 150 nucleotides in length. Sequencing was performed by the VANTAGE laboratory at Vanderbilt University Medical Center (Nashville, TN).

### Sequence Alignment and Filtering

Applicant aligned the sequences to the cattle reference genome (Bos_taurus.ARS-UCD1.2.105) in the Ensembl62 database with hisat2^{63,64,65} using the -very-sensitive parameter. Then, Applicant used Samtools^{66,67} to filter sequences and remove secondary alignments, duplicates, and unmapped reads. Next, Applicant used biobambam268 to mark and remove duplicates.

### Transcript Quantification and Gene Filtering

The number of fragments that matched to the Ensembl⁶² cow gene annotation (Bos_taurus.ARS-UCD1.2.105) was quantified using featureCounts⁶⁹, and Applicant preserved genes annotated as protein-coding, pseudogenes, or long non-coding RNA. Genes were then retained for further analysis if counts per million (CPM) and fragments per kilobase per million (FPKM) were >1 in at least five samples.

### Proteomics Data and Processing

One hundred μl of plasma per sample was submitted to the Virginia Tech Mass Spectrometry Incubator (VT-MSI) facility at the Fralin Life Sciences Institute, Virginia Tech, for protein extraction and data collection.

Plasma samples (100 μl) were acidified by the addition of 11.1 μl 12% (v/v) o-phosphoric acid (MilliporeSigma, St. Louis, MO), then proteins were precipitated by the addition of 725 μl LC/MS grade methanol and incubated at −80° C. overnight. Precipitated protein was collected by centrifugation and solubilized in S-trap lysis buffer (10% (w/v) SDS in 100 mM triethylammonium bicarbonate (MilliporeSigma, St. Louis, MO, pH 8.5)). Protein concentration was determined by measuring the absorbance at 280 nm, then 150 g of protein for each sample was reduced using DTT (4.5 mM) then alkylated with iodoacetamide (10 mM, MilliporeSigma, St. Louis, MO). Unreacted I iodoacetamide was quenched with DTT (10 mM, MilliporeSigma, St. Louis, MO) and samples were acidified using o-phosphoric acid (MilliporeSigma, St. Louis, MO). Protein was again precipitated using methanol and incubated at −80° C. overnight as above. Precipitated protein was loaded onto a micro S-trap and washed with methanol then digested overnight with trypsin. Peptides were recovered and five g, as determined by measuring the absorbance at 215 nm using a DS-11 FX+ spectrophotometer/fluorometer (DeNovix, Wilmington, DE), of each sample was analyzed twice (duplicates) using ESI-MS/MS Orbitrap Fusion Lumos (Thermo Fisher Scientific (Waltham, MA)).

Samples were first loaded onto a precolumn (Acclaim PepMap 100 (Thermo Scientific, Waltham, MA), 100 μm×2 cm) after which flow was diverted to an analytical column (50 cm μPAC (PharmaFluidics, Woburn, MA). The UPLC/autosampler utilized was an Easy-nLC 1200 (Thermo Scientific, Waltham, MA). Flow rate was maintained at 150 nl/min and peptides were eluted utilizing a 2 to 45% gradient of solvent B in solvent A over 88 min. Spray voltage on the μPAC compatible Easy-Spray emitter (PharmaFluidics, Woburn, MA) was 1300 volts, the ion transfer tube was maintained at 275° C., the RF lens was set to 30% and the default charge state was set to 3.

MS data for the m/z range of 400-1500 was collected using the orbitrap at 120,000 resolution in positive profile mode with an AGC target of 4.0e5 and a maximum injection time of 50 ms. Peaks were filtered for MS/MS analysis based on having isotopic peak distribution expected of a peptide with an intensity above 2.0e4 and a charge state of 2-5. Peaks were excluded dynamically for 15 s after 1 scan with the MS/MS set to be collected at 45% of a chromatographic peak width with an expected peak width (FWHM) of 15 s. MS/MS data starting at m/z of 150 was collected using the orbitrap at 15000 resolution in positive centroid mode with an AGC target of 1.0e5 and a maximum injection time of 200 ms. Activation type was HCD stepped from 27 to 33.

Data were analyzed utilizing Proteome Discoverer 2.5 (Thermo Scientific, Waltham, MA) combining a Sequest HT and Mascot 2.7 (Matrix Science, Boston, MA) search into one result summary for each sample. Both searches utilized the UniProt reference Bos taurus proteome database and a common protein contaminant database provided with the Proteome Discoverer (PD) software package⁷⁰. Each search assumed trypsin-specific peptides with the possibility of 2 missed cleavages, a precursor mass tolerance of 10 ppm and a fragment mass tolerance of 0.1 Da. Sequest HT searches also included the PD software precursor detector node to identify MS/MS spectra containing peaks from more than one precursor. Sequest HT searches included a fixed modification of carbamidomethyl at Cys and the variable modifications of oxidation at Met and loss of Met at the N-terminus of a protein (required for using the INFERYS rescoring node). Peptide matches identified by Sequest HT were subjected to INFERYS rescoring to further optimize the number of peptides identified with high confidence.

Mascot searches included the following dynamic modifications in addition to the fixed modification of Cys alkylated by iodoacetamide (carbamidomethylated): oxidation of Met, acetylation of the protein N-terminus, cyclization of a peptide N-terminal Gln to pyro-Glu, and deamidation of Asn/Gln residues.

Protein identifications were reported at a 1% false discovery rate (high confidence) or at 5% false discovery rate (medium confidence) based on searches of decoy databases utilizing the same parameters as above. The software matched peptide peaks across all runs, and protein quantities are the sum of all peptide intensities associated with the protein.

### Principal Component Analysis

Applicant carried out principal component analysis for the genotypes after pruning using the parameter ‘--pca’ in PLINK. The eigenvectors were used for plotting. For the transcriptome data, first Applicant obtained the variant stabilized data using the function ‘vst’ from the R package ‘DESeq2’. Next, Applicant calculated the components using the function ‘plotPCA’ in R. For the protein data, Applicant averaged the values for each technical duplicate and used these values as input for the function ‘prcomp’ in R.

### Statistical Analyses

### SNP Association Analysis

After filtering, 575,053 genotypes from 22 animals were used for association analysis conducted in PLINK⁶⁰ using Fisher's exact test. Applicant adjusted the nominal P values to correct for multiple hypothesis testing using the adaptative permutation procedure⁷¹ in PLINK⁶⁰. Locus association was inferred at alpha=1×10⁻⁵, as reported by The Wellcome Trust Case Control Consortium⁷² for case-control studies, as well as by previous GWAS analyses of reproductive traits in cows or heifers^{4,25,32}, which corresponded to an adjusted P value<0.005.

### Differential Transcript Abundance

Applicant compared transcript abundance between samples from each breed and each fertility group. The R packages ‘edgeR’^{73,74}, with the quasi-likelihood test, and ‘DEseq2’⁷⁵, using the Wald's and likelihood test, were utilized to conduct the analyses. Applicant adjusted the raw P values for multiple hypothesis testing by calculating the empirical false discovery rate (eFDR⁷⁶), with 10,000 permutations. Differences in transcript abundance were deemed statistically significant when eFDR<0.002 in the results obtained from the three tests.

### Differential Protein Abundance

To identify differential protein abundance that is robust to the algorithm utilized, Applicant analyzed the protein data using two different algorithms. First, we transformed the protein data using natural logarithm (Logₑ(x)). Applicant analyzed the transformed data using a generalized mixed model⁷⁷ using the R package ‘lme4’, which included the fertility group (Fertile or Sub-Fertile), breed (Angus or Holstein), and the random effect of the subject. Random effect was included in this analysis as samples were assayed twice to provide a more robust estimate of differential protein abundance. Then, Applicant used the function ‘emmeans’, which tests the significance of the difference (H₀:μ₁=μ₂, H₁:μ₁≠μ₂) with the Student's t test⁷⁸, to calculate the estimated differences in protein abundance between fertility groups within each breed. Applicant also analyzed the log-transformed data using the R package ‘limma’⁷⁹. Applicant accounted for the same independent variables mentioned above (fertility group and breed), in addition to accounting for the correlation between the duplicated data for each individual with the function ‘duplicateCorrelation’. Applicant tested for a differential abundance of the identified proteins using the empirical Bayes Statistics implemented in the function ‘eBayes’^{80,81}. In both analyses, Applicant adjusted the nominal P values using FDR⁸². Significance was assumed if FDR<0.05 in both approaches.

### Multi-Omics Factor Analysis

Applicant analyzed the multimodal multi-omics datasets (genome, transcriptome, and proteome) interactively using Multi-omics Factor Analysis approach^{83,84}. Applicant subset the genotypes, transcriptome, and proteome data to reduce the global profiling. Applicant retained SNPs with a Pvalue <0.001 for the Fisher's test, genes with a P value <0.01 for all three statistical tests employed, and proteins with a P value <0.05 in both statistical tests used. Applicant conducted the analysis using the R package ‘MOFA2’^{83,84}, accounting for the breed as a group.

### Results

### Overview of the Data Produced

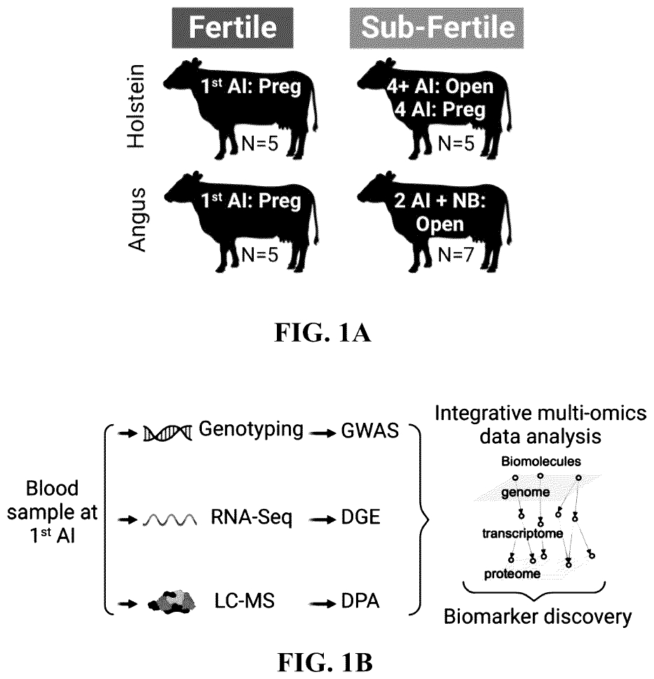

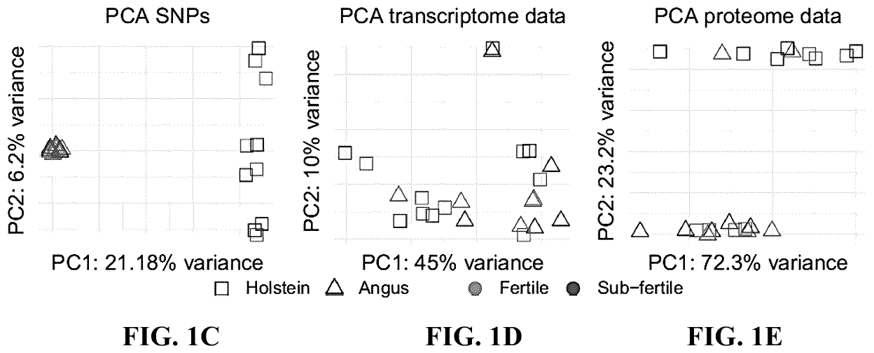

Applicant selected 22 Bos taurus heifers of Angus (n=12) and Holstein (n=10) breeds based on their fertility fitness (FIG. 1A). Applicant isolated total RNA from circulating white blood cells, averaging 16.3 μg±4.0, and quality, measured by the RIN, averaging 9.4±0.4. The extraction of genomic DNA yielded 1.1 μg±0.4. Applicant produced RNA-sequencing data (FIG. 1B) and quantified the transcript abundance of 12,445 genes (12,105 protein-coding genes, 228 long non-coding RNAs, and 112 pseudogenes). Applicant also analyzed 575,053 nucleotide positions across the bovine genome (FIG. 1B). Lastly, Applicant produced untargeted proteomics data from plasma that resulted in the relative quantification of 213 proteins. As expected, the genotypic and proteomic data clustered the heifers of different genetic background separately (FIG. 1A). Conversely, there was no clustering of the samples based on the transcriptome data of the peripheral white blood cells (FIG. 1D,E).

GWAS Identifies SNPs Associated with Fertility in Angus and Holsteins Heifers

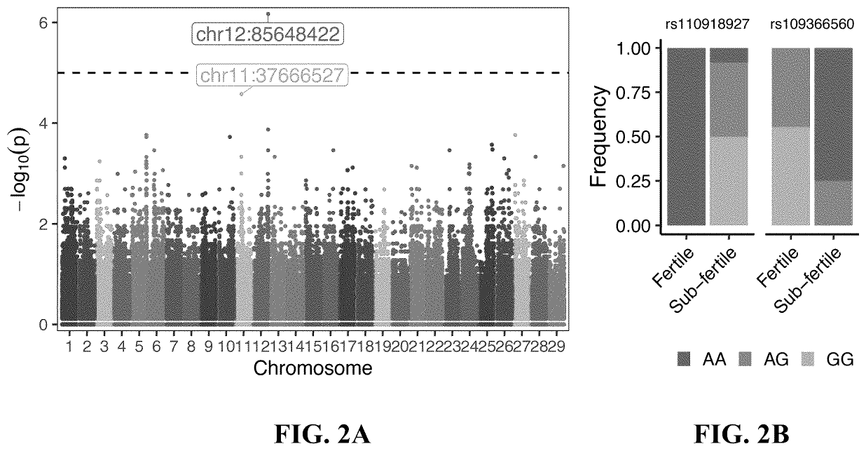

Applicant's analysis identified two SNPs significantly associated with heifer fertility (rs110918927, chr12: 85648422, P=6.7×10-7; and rs109366560, chr11:37666527, P=2.6×10-5, FIG. 2A, Supplementary information 3 of Marella and Biase. “A multi-omics analysis identifies molecular features associated with fertility in heifers (Bos taurus)” Sci. Rep. (2023) 13:12664, which is hereby incorporated by reference as if expressed in its entirety herein). For the SNP rs110918927, all heifers that delivered a calf after one artificial insemination presented the genotype AA (f(A)=1, f(G)=0), whereas 11 out of 12 heifers classified as sub-fertile presented at least one copy of the allele G (f(A)=0.29, f(G)=0.71). This polymorphism sits in an intergenic region of the genome with the closest gene located >73 kilobases downstream relative to the SNP. For the SNP rs109366560, none of the heifers classified as sub-fertile were homozygous for the allele G (f(G)=0.12, f(A)=0.88), and five out of the nine fertile heifers genotyped were homozygous GG (f(G)=0.78, f(A)=0.22). This SNP is located on intron 22 of the gene Echinoderm microtubule associated protein like 6 (EML6).

### Transcriptome Analysis Identifies Differential Transcript Abundance Between Fertile and Sub-Fertile Heifers

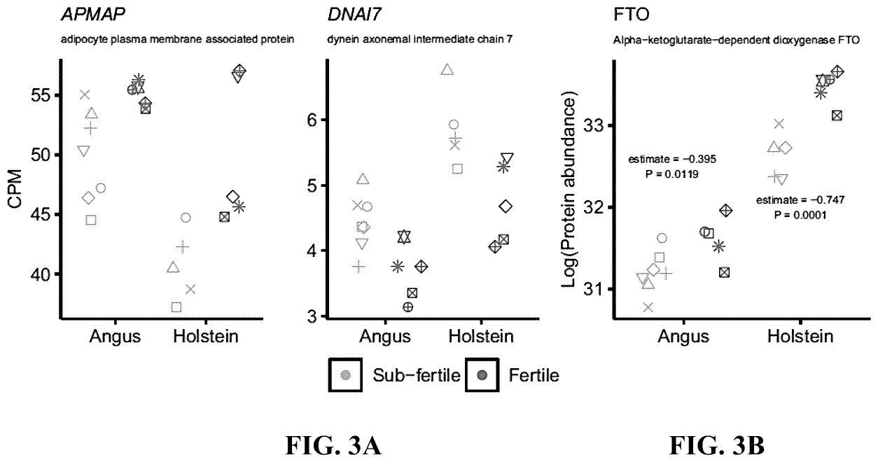

Next, Applicant sought to determine if there were differences in transcript abundance from circulating white blood cells between the Fertile and Sub-Fertile heifer groups, accounting for their genetic background. Applicant identified two genes whose transcript abundance differed (eFDR≤0.002) between the two groups (Fertile and Sub-Fertile), namely Adipocyte Plasma Membrane Associated Protein (APMAP, 1.16 greater abundance in the Fertile group) and Dynein Axonemal Intermediate Chain 7 (DNAI7, 1.23 greater abundance in the Sub-Fertile group) (FIG. 3A, Supplementary Information 4 of Marella and Biase. “A multi-omics analysis identifies molecular features associated with fertility in heifers (Bos taurus)” Sci. Rep. (2023) 13:12664, which is hereby incorporated by reference as if expressed in its entirety herein).

### Proteomic Analysis Identifies Differential Protein Abundance Between Fertile and Sub-Fertile Heifers

Applicant also tested if there were differential abundance in proteins present in the plasma of heifers classified based on their fertility groups in both genetic backgrounds. The protein Alpha-ketoglutarate-dependent dioxygenase FTO was more abundant in the plasma collected from Fertile heifers relative to their Sub-Fertile counterparts (FDR<0.05, FIG. 3B, Table 2).

TABLE 2

| | General linear mixed model | | | | | | |
|---|---|---|---|---|---|---|---|
| protein | F_stat | p_value | contrast | estimate | SE | p_value_t | fdr |
| A5D798 | 24.9151338 | 9.46E−05 | NP - P | −0.5608937 | 0.11236963 | 9.46E−05 | 0.02014218 |
| P19034 | 18.0382217 | 0.00048473 | NP - P | 1.43451075 | 0.33775901 | 0.00048473 | 0.05162341 |
| F1MYX5 | 15.6183702 | 0.00093459 | NP - P | 1.11069492 | 0.28104569 | 0.00093459 | 0.06635565 |
| P01035 | 10.1596534 | 0.00510159 | NP - P | 1.37101254 | 0.43013222 | 0.00510159 | 0.24757402 |
| F1N102 | 9.78455469 | 0.0058116 | NP - P | 0.71422331 | 0.22833027 | 0.0058116 | 0.24757402 |
| P02768 | 8.50654652 | 0.00920907 | NP - P | 0.62483954 | 0.21423571 | 0.00920907 | 0.29346188 |
| G3MY71 | 8.38233886 | 0.00964429 | NP - P | −1.0384737 | 0.35868474 | 0.00964429 | 0.29346188 |
| P00744 | 7.7521001 | 0.01224251 | NP - P | 1.22243566 | 0.43905237 | 0.01224251 | 0.32595677 |
| A0A6B9SCM2 | 7.09288063 | 0.01583892 | NP - P | 0.84812152 | 0.31845404 | 0.01583892 | 0.36059061 |
| Q5E9E3 | 6.64325472 | 0.01897695 | NP - P | 0.3980051 | 0.15441809 | 0.01897695 | 0.36059061 |
| Q2HJF0 | 6.27834362 | 0.02204691 | NP - P | 0.41467688 | 0.16549592 | 0.02204691 | 0.36059061 |
| P80109 | 6.22493354 | 0.02254187 | NP - P | 0.58489367 | 0.23442804 | 0.02254187 | 0.36059061 |
| Q95121 | 6.16227613 | 0.02313867 | NP - P | 0.29877505 | 0.12035766 | 0.02313867 | 0.36059061 |
| G1K122 | 6.05376932 | 0.02421513 | NP - P | 0.29708932 | 0.12074638 | 0.02421513 | 0.36059061 |
| P17690 | 5.87925015 | 0.02606803 | NP - P | 0.26811558 | 0.11057605 | 0.02606803 | 0.36059061 |
| G5E5V0 | 5.78921876 | 0.02708662 | NP - P | −0.4286096 | 0.17813607 | 0.02708662 | 0.36059061 |
| A0A6B9SE58 | 5.51597712 | 0.03046659 | NP - P | 1.13831477 | 0.48467559 | 0.03046659 | 0.37140958 |
| Q5EA67 | 5.36676666 | 0.03251392 | NP - P | −0.5344919 | 0.23071974 | 0.03251392 | 0.37140958 |
| P06868 | 5.29655093 | 0.03353138 | NP - P | 0.52093328 | 0.22635262 | 0.03353138 | 0.37140958 |
| A0A6B9SEF7 | 5.20752225 | 0.03487414 | NP - P | −0.4728322 | 0.2072008 | 0.03487414 | 0.37140958 |
| F6QND5 | 5.09681727 | 0.03663039 | NP - P | 0.1347622 | 0.05969233 | 0.03663039 | 0.3715368 |
| F1N0I3 | 4.78907294 | 0.04206941 | NP - P | −0.5190372 | 0.23717709 | 0.04206941 | 0.40730842 |
| Q2KIU3 | 4.67311691 | 0.04435483 | NP - P | −0.1514531 | 0.07006075 | 0.04435483 | 0.41076433 |
| A0A3Q1MI29 | 4.43024433 | 0.04961997 | NP - P | −0.7760961 | 0.36872424 | 0.04961997 | 0.42425279 |
| Q2KIX7 | 4.33126356 | 0.05196958 | NP - P | 0.24565959 | 0.11803924 | 0.05196958 | 0.42425279 |
| E1BF81 | 4.32165302 | 0.05220447 | NP - P | 0.47403924 | 0.22802861 | 0.05220447 | 0.42425279 |
| G3X6N3 | 4.12390658 | 0.05732058 | NP - P | 0.35974645 | 0.17715039 | 0.05732058 | 0.42425279 |
| A0A6B9SBF6 | 4.09219091 | 0.05819396 | NP - P | 0.56914897 | 0.28135071 | 0.05819396 | 0.42425279 |
| F1MZ96 | 4.09136953 | 0.05821678 | NP - P | −0.3934163 | 0.19449926 | 0.05821678 | 0.42425279 |
| A5PJ69 | 4.03689027 | 0.05975391 | NP - P | 0.6408532 | 0.31895916 | 0.05975391 | 0.42425279 |
| A0A3Q1MS75 | 3.79830445 | 0.06706446 | NP - P | 0.4633378 | 0.23774032 | 0.06706446 | 0.43884579 |
| B8Y9T0 | 3.78556959 | 0.06748299 | NP - P | 0.50317192 | 0.25861325 | 0.06748299 | 0.43884579 |
| E1B805 | 3.77025913 | 0.06799019 | NP - P | 0.18044766 | 0.09293208 | 0.06799019 | 0.43884579 |
| Q2KJH6 | 3.69931141 | 0.07039918 | NP - P | 0.36008639 | 0.18721742 | 0.07039918 | 0.44036557 |
| A0A3Q1MNN6 | 3.57514843 | 0.07485851 | NP - P | −0.253977 | 0.13432205 | 0.07485851 | 0.44036557 |
| Q2KIF2 | 3.56127108 | 0.0753771 | NP - P | 0.18250862 | 0.09671211 | 0.0753771 | 0.44036557 |
| A0A3Q1MGP1 | 3.53172016 | 0.07649543 | NP - P | −0.22866 | 0.12167378 | 0.07649543 | 0.44036557 |
| A0A3Q1LI44 | 3.36451279 | 0.08320113 | NP - P | −0.3426325 | 0.18679594 | 0.08320113 | 0.45782454 |
| P02676 | 3.34971589 | 0.08382703 | NP - P | 0.3414556 | 0.18656503 | 0.08382703 | 0.45782454 |
| ENSEMBL:ENSBTAP00000038329 | 3.19915602 | 0.09052024 | NP - P | 0.46903598 | 0.26223366 | 0.09052024 | 0.48202028 |
| A0A3Q1LQ21 | 3.04774786 | 0.09789452 | NP - P | 0.47397546 | 0.27149782 | 0.09789452 | 0.50252831 |
| F1MAV0 | 3.02273222 | 0.09918007 | NP - P | −0.1985162 | 0.1141816 | 0.09918007 | 0.50252831 |
| A0A6B9SBI8 | 2.97949577 | 0.10144938 | NP - P | 0.43434686 | 0.25163167 | 0.10144938 | 0.50252831 |
| Q7SIH1 | 2.89825191 | 0.10588209 | NP - P | 0.11449795 | 0.06725579 | 0.10588209 | 0.51015678 |
| Q68RU0 | 2.864698 | 0.1077796 | NP - P | −1.2040386 | 0.71137894 | 0.1077796 | 0.51015678 |
| P55906 | 2.82196268 | 0.11025515 | NP - P | 0.89011173 | 0.52986946 | 0.11025515 | 0.51052929 |
| A0A3Q1ML26 | 2.71322348 | 0.11686577 | NP - P | 0.38136867 | 0.23152731 | 0.11686577 | 0.51941586 |
| A6QPQ2 | 2.48060397 | 0.13267026 | NP - P | −0.3173386 | 0.20148567 | 0.13267026 | 0.51941586 |
| A0A3Q1LI40 | 2.45015705 | 0.13492394 | NP - P | 0.20595151 | 0.13157338 | 0.13492394 | 0.51941586 |
| A0A140T897 | 2.38541782 | 0.13987147 | NP - P | 0.08464157 | 0.05480264 | 0.13987147 | 0.51941586 |
| A0A6B9SBL4 | 2.38229264 | 0.1401158 | NP - P | 0.91805284 | 0.59479885 | 0.1401158 | 0.51941586 |
| A0A3Q1M5R4 | 2.35325445 | 0.14241097 | NP - P | 1.04383137 | 0.6804495 | 0.14241097 | 0.51941586 |
| A0A6B9SCG2 | 2.34061071 | 0.14342455 | NP - P | 0.96952867 | 0.63371803 | 0.14342455 | 0.51941586 |
| A0A3Q1MIL5 | 2.31824486 | 0.14523901 | NP - P | −0.5562316 | 0.36532216 | 0.14523901 | 0.51941586 |
| F1MS32 | 2.3092487 | 0.14597668 | NP - P | −0.5743599 | 0.37796253 | 0.14597668 | 0.51941586 |
| A0A6B9SBU4 | 2.3063509 | 0.14621527 | NP - P | 0.25124772 | 0.16543959 | 0.14621527 | 0.51941586 |
| A0A6B9SDY9 | 2.3044335 | 0.14637339 | NP - P | 0.17245191 | 0.11360199 | 0.14637339 | 0.51941586 |
| P00761 | 2.30174732 | 0.14659527 | NP - P | 0.49328392 | 0.32513831 | 0.14659527 | 0.51941586 |
| A0A6B9SDP2 | 2.29626398 | 0.14704945 | NP - P | −0.4571086 | 0.30165355 | 0.14704945 | 0.51941586 |
| K4JB97 | 2.28349239 | 0.14811398 | NP - P | 1.33972351 | 0.88657483 | 0.14811398 | 0.51941586 |
| A6QP30 | 2.26627392 | 0.14956403 | NP - P | 0.23056116 | 0.15315456 | 0.14956403 | 0.51941586 |
| A0A3Q1NJR8 | 2.22573006 | 0.15304728 | NP - P | 0.18500908 | 0.12401003 | 0.15304728 | 0.51941586 |
| P01888 | 2.21905679 | 0.15363004 | NP - P | −0.3183751 | 0.21372477 | 0.15363004 | 0.51941586 |
| I7CT57 | 2.17035869 | 0.15796578 | NP - P | 0.12128522 | 0.08232697 | 0.15796578 | 0.52572987 |
| Q3MHH8 | 2.13821581 | 0.16090969 | NP - P | −0.9909214 | 0.67766249 | 0.16090969 | 0.52728868 |
| G3N3S2 | 2.07203185 | 0.16718624 | NP - P | 0.29920069 | 0.20785687 | 0.16718624 | 0.5316066 |
| Q28085 | 2.03599499 | 0.17073044 | NP - P | 0.21221301 | 0.14872489 | 0.17073044 | 0.5316066 |
| Q29437 | 2.03271969 | 0.17105713 | NP - P | 0.115885 | 0.0812809 | 0.17105713 | 0.5316066 |
| P34955 | 2.02121703 | 0.17221059 | NP - P | 0.16879503 | 0.118728 | 0.17221059 | 0.5316066 |
| A0A6B9SCC1 | 1.95695848 | 0.17883415 | NP - P | −0.464442 | 0.33200196 | 0.17883415 | 0.54416677 |
| A0A6B9SBM2 | 1.77581087 | 0.19928933 | NP - P | 0.33816654 | 0.25376532 | 0.19928933 | 0.59131735 |
| A0A6B9SDM9 | 1.75220554 | 0.20216623 | NP - P | −0.7187091 | 0.54295098 | 0.20216623 | 0.59131735 |
| A0A3Q1NEQ0 | 1.74821239 | 0.20265806 | NP - P | 0.21754044 | 0.16452917 | 0.20265806 | 0.59131735 |
| F6R7W8 | 1.71760104 | 0.20647894 | NP - P | −0.3614436 | 0.27579052 | 0.20647894 | 0.59432451 |
| A0A6B9SBT1 | 1.69398961 | 0.20948844 | NP - P | −0.5538693 | 0.42555131 | 0.20948844 | 0.59494716 |
| P41361 | 1.65045222 | 0.21518491 | NP - P | 0.16072607 | 0.12510793 | 0.21518491 | 0.60308403 |
| Q29443 | 1.57975215 | 0.22486225 | NP - P | −0.2574332 | 0.20481895 | 0.22486225 | 0.61302687 |
| P13645 | 1.57515821 | 0.22551013 | NP - P | 0.48657132 | 0.38768984 | 0.22551013 | 0.61302687 |
| G3N0V0 | 1.56208637 | 0.22736677 | NP - P | −0.1866109 | 0.14930845 | 0.22736677 | 0.61302687 |
| Q28107 | 1.51169643 | 0.23471033 | NP - P | −0.1526296 | 0.12413847 | 0.23471033 | 0.62491626 |
| G3MYZ3 | 1.45735213 | 0.24297693 | NP - P | 0.2742998 | 0.22721826 | 0.24297693 | 0.63893935 |
| A0A6B9SBJ6 | 1.4093977 | 0.2505881 | NP - P | −0.3547 | 0.2987751 | 0.2505881 | 0.64165172 |
| ENSEMBL:ENSBTAP00000014147 | 1.40261752 | 0.25168919 | NP - P | −0.1559397 | 0.13167011 | 0.25168919 | 0.64165172 |
| ENSEMBL:ENSBTAP00000031900 | 1.39431743 | 0.25304575 | NP - P | 0.09462629 | 0.08013661 | 0.25304575 | 0.64165172 |
| B0JYQ0 | 1.3242561 | 0.26488804 | NP - P | 0.14697707 | 0.12772138 | 0.26488804 | 0.66377827 |
| A0A3Q1LKB0 | 1.29903534 | 0.26932974 | NP - P | −0.4026692 | 0.35329536 | 0.26932974 | 0.66407535 |
| F1MLW8 | 1.28809412 | 0.27128735 | NP - P | −0.364753 | 0.32138461 | 0.27128735 | 0.66407535 |
| A0A6B9SCN1 | 1.25145782 | 0.27798183 | NP - P | −0.2339724 | 0.20914937 | 0.27798183 | 0.66407535 |
| A0A0A0MP92 | 1.24804223 | 0.27861714 | NP - P | 0.13398932 | 0.11993765 | 0.27861714 | 0.66407535 |
| A6QPP2 | 1.23747397 | 0.28059522 | NP - P | 0.22797208 | 0.20493381 | 0.28059522 | 0.66407535 |
| Q1RMH5 | 1.21201089 | 0.28543914 | NP - P | 0.32393909 | 0.29424568 | 0.28543914 | 0.6681158 |
| A0A3Q1LJT1 | 1.18883419 | 0.28994642 | NP - P | −0.4827981 | 0.44279727 | 0.28994642 | 0.67128899 |
| A0A3Q1N1A7 | 1.14159239 | 0.29943638 | NP - P | −0.2055053 | 0.19233903 | 0.29943638 | 0.67618453 |
| A0A6B9SBI1 | 1.11013181 | 0.30599206 | NP - P | 0.46071777 | 0.437268 | 0.30599206 | 0.67618453 |
| A0A6B9SBJ3 | 1.1032781 | 0.30744624 | NP - P | 0.22612365 | 0.2152799 | 0.30744624 | 0.67618453 |
| A2I7N0 | 1.10319208 | 0.30746455 | NP - P | 0.17235146 | 0.16409275 | 0.30746455 | 0.67618453 |
| P17697 | 1.09345268 | 0.30954753 | NP - P | 0.20275467 | 0.19389691 | 0.30954753 | 0.67618453 |
| Q3SYR0 | 1.07951878 | 0.3125615 | NP - P | −0.1422675 | 0.13692749 | 0.3125615 | 0.67618453 |
| A0A6B9SDW4 | 1.07164318 | 0.31428295 | NP - P | −0.4177598 | 0.40355386 | 0.31428295 | 0.67618453 |
| Q3SZV7 | 1.0188179 | 0.32617644 | NP - P | 0.09137448 | 0.09052669 | 0.32617644 | 0.67956723 |
| A5PK72 | 1.01777341 | 0.32641788 | NP - P | 0.3856203 | 0.38223842 | 0.32641788 | 0.67956723 |
| A0A3Q1M3L6 | 1.01305517 | 0.32751164 | NP - P | 0.93884528 | 0.93277625 | 0.32751164 | 0.67956723 |
| P01045-2 | 1.0083092 | 0.32861702 | NP - P | 0.5071197 | 0.50502586 | 0.32861702 | 0.67956723 |
| Q9TTE1 | 0.97053654 | 0.33760444 | NP - P | 0.14661243 | 0.14882121 | 0.33760444 | 0.69143985 |
| P35527 | 0.90747432 | 0.35340352 | NP - P | 0.47671839 | 0.50043161 | 0.35340352 | 0.71291545 |
| F1MKS5 | 0.90215513 | 0.35478422 | NP - P | 0.29856009 | 0.31433384 | 0.35478422 | 0.71291545 |
| A0A6B9SBR2 | 0.87427923 | 0.36214884 | NP - P | 0.4336695 | 0.46380327 | 0.36214884 | 0.72091311 |
| E1BCW0 | 0.85549668 | 0.36723677 | NP - P | 0.29229174 | 0.31601477 | 0.36723677 | 0.72427252 |
| F1MJ12 | 0.8320659 | 0.37373155 | NP - P | −0.4011272 | 0.4397474 | 0.37373155 | 0.73031946 |
| Q95122 | 0.79350112 | 0.38479586 | NP - P | 0.33228949 | 0.3730292 | 0.38479586 | 0.74168048 |
| Q2KIT0 | 0.78596273 | 0.38701541 | NP - P | 0.13286838 | 0.14987206 | 0.38701541 | 0.74168048 |
| P02672 | 0.77595534 | 0.38999161 | NP - P | −0.1870304 | 0.2123214 | 0.38999161 | 0.74168048 |
| ENSEMBL:ENSBTAP00000016046 | 0.74862782 | 0.39829632 | NP - P | −0.1990321 | 0.23003297 | 0.39829632 | 0.74863067 |
| A0A6B9SBM1 | 0.74095683 | 0.40067557 | NP - P | −0.4832117 | 0.56135942 | 0.40067557 | 0.74863067 |
| A0A3Q1NJB1 | 0.71251128 | 0.40969009 | NP - P | 0.08947902 | 0.10600475 | 0.40969009 | 0.75881729 |
| P28800 | 0.69748626 | 0.41457777 | NP - P | 0.08049456 | 0.09638261 | 0.41457777 | 0.75914597 |
| Q32PA1 | 0.69015429 | 0.41699567 | NP - P | 0.32006778 | 0.38527326 | 0.41699567 | 0.75914597 |
| E1B7G5 | 0.65860736 | 0.42765387 | NP - P | 0.17477751 | 0.21536357 | 0.42765387 | 0.77195148 |
| E1BH06 | 0.61690091 | 0.44241774 | NP - P | −0.1346994 | 0.17149759 | 0.44241774 | 0.78873983 |
| A0A6B9SCH7 | 0.61157811 | 0.44436047 | NP - P | 0.29712017 | 0.37993226 | 0.44436047 | 0.78873983 |
| A0A6B9SCJ6 | 0.58146631 | 0.45561546 | NP - P | −0.3351676 | 0.4395414 | 0.45561546 | 0.79717021 |
| A0A6B9SE69 | 0.57411893 | 0.45843239 | NP - P | 0.18389557 | 0.24270031 | 0.45843239 | 0.79717021 |
| A0A0A0MPA0 | 0.5691912 | 0.46033772 | NP - P | 0.48200033 | 0.63887846 | 0.46033772 | 0.79717021 |
| Q17QC8 | 0.54309272 | 0.47065118 | NP - P | 0.19013471 | 0.2580028 | 0.47065118 | 0.80102898 |
| Q32L76 | 0.53034133 | 0.47583148 | NP - P | 0.4551889 | 0.6250488 | 0.47583148 | 0.80102898 |
| A0A6B9SE85 | 0.52510976 | 0.4779847 | NP - P | 0.29788019 | 0.41107075 | 0.4779847 | 0.80102898 |
| K4JDT2 | 0.5195827 | 0.48027755 | NP - P | 0.25376368 | 0.3520482 | 0.48027755 | 0.80102898 |
| Q2KI67 | 0.51696616 | 0.48136953 | NP - P | −0.3055771 | 0.42500083 | 0.48136953 | 0.80102898 |
| G3N1H5 | 0.49855694 | 0.48917393 | NP - P | 0.42693102 | 0.60464481 | 0.48917393 | 0.80334217 |
| E1BH94 | 0.49593603 | 0.49030273 | NP - P | 0.29866435 | 0.42410222 | 0.49030273 | 0.80334217 |
| A5PKC2 | 0.45290499 | 0.50950733 | NP - P | −0.1620859 | 0.24084726 | 0.50950733 | 0.82843559 |
| A0A6B9SDY7 | 0.44288407 | 0.51417183 | NP - P | −0.0905471 | 0.13605969 | 0.51417183 | 0.82968635 |
| A0A6B9SBR8 | 0.41659939 | 0.52678155 | NP - P | 0.32686001 | 0.50641024 | 0.52678155 | 0.84364263 |
| C4T8B4 | 0.37798069 | 0.54638009 | NP - P | 0.16805875 | 0.27335455 | 0.54638009 | 0.86849969 |
| P01030 | 0.36297883 | 0.55437139 | NP - P | −0.248615 | 0.41265467 | 0.55437139 | 0.87079648 |
| A0A6B9SDY5 | 0.35237696 | 0.56015705 | NP - P | 0.15094853 | 0.25428757 | 0.56015705 | 0.87079648 |
| Q9TS85 | 0.3523408 | 0.56017699 | NP - P | 0.15267419 | 0.25720781 | 0.56017699 | 0.87079648 |
| A0A3Q1LPG0 | 0.3451347 | 0.564178 | NP - P | 0.30776594 | 0.52387328 | 0.564178 | 0.87079648 |
| Q3T004 | 0.33426488 | 0.57032179 | NP - P | −0.2712028 | 0.46908199 | 0.57032179 | 0.87394634 |
| O02659 | 0.31406224 | 0.58210689 | NP - P | −0.1739233 | 0.31034875 | 0.58210689 | 0.88563406 |
| G3N0S9 | 0.26118642 | 0.6155177 | NP - P | −0.0866307 | 0.1695105 | 0.6155177 | 0.91085108 |
| A0A452DHX8 | 0.25996903 | 0.61633624 | NP - P | 0.09745218 | 0.19113082 | 0.61633624 | 0.91085108 |
| A0A3Q1MIN7 | 0.25808403 | 0.6176085 | NP - P | −0.1663796 | 0.32750627 | 0.6176085 | 0.91085108 |
| V6F9A3 | 0.25686762 | 0.61843262 | NP - P | 0.11503636 | 0.22697627 | 0.61843262 | 0.91085108 |
| A0A6B9SE14 | 0.25447199 | 0.62006294 | NP - P | 0.2262858 | 0.44857731 | 0.62006294 | 0.91085108 |
| Q58DL9 | 0.24305497 | 0.62796875 | NP - P | 0.19143181 | 0.38829505 | 0.62796875 | 0.91469881 |
| A5D7Q2 | 0.23838312 | 0.63127101 | NP - P | 0.25655729 | 0.52546841 | 0.63127101 | 0.91469881 |
| A0A3Q1MJT2 | 0.22836278 | 0.63849238 | NP - P | 0.07338437 | 0.15356453 | 0.63849238 | 0.91891134 |
| G5E5A8 | 0.20433419 | 0.65664482 | NP - P | −0.2010344 | 0.44473343 | 0.65664482 | 0.9264515 |
| A0A6B9SF17 | 0.20313028 | 0.65758781 | NP - P | −0.3303926 | 0.73306579 | 0.65758781 | 0.9264515 |
| P15497 | 0.19447907 | 0.66446572 | NP - P | −0.0428316 | 0.0971243 | 0.66446572 | 0.9264515 |
| Q3T101 | 0.19292631 | 0.6657196 | NP - P | −0.105499 | 0.24018861 | 0.6657196 | 0.9264515 |
| A5PK49 | 0.1869214 | 0.67062652 | NP - P | 0.20097992 | 0.46486104 | 0.67062652 | 0.9264515 |
| F1MMK9 | 0.18195445 | 0.67475697 | NP - P | −0.1544529 | 0.36208853 | 0.67475697 | 0.9264515 |
| A0A6B9SDR8 | 0.17695098 | 0.6789862 | NP - P | −0.1008508 | 0.23974677 | 0.6789862 | 0.9264515 |
| P04264 | 0.16858748 | 0.68621656 | NP - P | 0.16551639 | 0.40311443 | 0.68621656 | 0.9264515 |
| Q2KIS7 | 0.16397816 | 0.69029194 | NP - P | 0.03460142 | 0.08544781 | 0.69029194 | 0.9264515 |
| A0A452DK44 | 0.16019131 | 0.69369062 | NP - P | −0.1559817 | 0.38972121 | 0.69369062 | 0.9264515 |
| A0A452DHZ3 | 0.1581555 | 0.69553718 | NP - P | 0.05569703 | 0.14005217 | 0.69553718 | 0.9264515 |
| A0A452DJ14 | 0.15275041 | 0.70050796 | NP - P | −0.0527323 | 0.13492294 | 0.70050796 | 0.9264515 |
| E1BMJ0 | 0.14512073 | 0.70770131 | NP - P | 0.08860934 | 0.23260272 | 0.70770131 | 0.9264515 |
| A0A6B9SC93 | 0.14424332 | 0.70854241 | NP - P | −0.3176832 | 0.8364624 | 0.70854241 | 0.9264515 |
| A0A452DI66 | 0.13771669 | 0.71489331 | NP - P | 0.03927884 | 0.10584379 | 0.71489331 | 0.9264515 |
| A0A6B9SDW5 | 0.13184726 | 0.72075348 | NP - P | −0.1491063 | 0.41063919 | 0.72075348 | 0.9264515 |
| E1B726 | 0.13077558 | 0.72183944 | NP - P | 0.03088193 | 0.0853967 | 0.72183944 | 0.9264515 |
| Q3SZZ9 | 0.12823362 | 0.72443562 | NP - P | 0.02377435 | 0.06639077 | 0.72443562 | 0.9264515 |
| P12763 | 0.12635534 | 0.72637277 | NP - P | 0.08184784 | 0.23025572 | 0.72637277 | 0.9264515 |
| Q3MHN2 | 0.10792562 | 0.74630931 | NP - P | −0.0649939 | 0.19783837 | 0.74630931 | 0.94621359 |
| ENSEMBL:ENSBTAP00000007350 | 0.09548422 | 0.76086564 | NP - P | 0.09901251 | 0.32042343 | 0.76086564 | 0.94773035 |
| A0A3B0J3V0 | 0.0941212 | 0.76252258 | NP - P | 0.06979499 | 0.22749954 | 0.76252258 | 0.94773035 |
| D4QBB4 | 0.09301894 | 0.76387218 | NP - P | −0.1389914 | 0.45572451 | 0.76387218 | 0.94773035 |
| Q1RMN8 | 0.09185782 | 0.76530338 | NP - P | 0.13679124 | 0.45133624 | 0.76530338 | 0.94773035 |
| Q2KJ63 | 0.08426901 | 0.77491129 | NP - P | −0.0348286 | 0.11997826 | 0.77491129 | 0.95152276 |
| P60712 | 0.08138862 | 0.77868108 | NP - P | −0.0822449 | 0.28828853 | 0.77868108 | 0.95152276 |
| A0A452DIP8 | 0.07656852 | 0.78515509 | NP - P | −0.1039135 | 0.37553201 | 0.78515509 | 0.95152276 |
| A0A6B9SE50 | 0.07578013 | 0.78623477 | NP - P | 0.11347678 | 0.41222026 | 0.78623477 | 0.95152276 |
| Q5EAD3 | 0.06651719 | 0.79940257 | NP - P | 0.04806067 | 0.1863472 | 0.79940257 | 0.95218874 |
| A0A6B9SBG6 | 0.0583083 | 0.8119194 | NP - P | 0.06176523 | 0.25578726 | 0.8119194 | 0.95218874 |
| O46375 | 0.05047213 | 0.82477432 | NP - P | −0.0268395 | 0.11946707 | 0.82477432 | 0.95218874 |
| A0A6B9SBF3 | 0.05006351 | 0.82547265 | NP - P | 0.1620936 | 0.72444469 | 0.82547265 | 0.95218874 |
| A0A3Q1ME55 | 0.04699229 | 0.83082007 | NP - P | −0.0790446 | 0.36463557 | 0.83082007 | 0.95218874 |
| G5E5T5 | 0.04606292 | 0.8324742 | NP - P | −0.0614346 | 0.28624468 | 0.8324742 | 0.95218874 |
| A0A3Q1M5Q6 | 0.04438562 | 0.8355044 | NP - P | −0.0755914 | 0.35879885 | 0.8355044 | 0.95218874 |
| G3N1U4 | 0.04396178 | 0.83627957 | NP - P | 0.04532478 | 0.21617133 | 0.83627957 | 0.95218874 |
| F1MDS0 | 0.04312798 | 0.83781599 | NP - P | 0.0993858 | 0.47856924 | 0.83781599 | 0.95218874 |
| P01017 | 0.04243973 | 0.83909594 | NP - P | 0.04144041 | 0.2011582 | 0.83909594 | 0.95218874 |
| Q09TE3 | 0.03984533 | 0.84402098 | NP - P | −0.0341934 | 0.17129863 | 0.84402098 | 0.95218874 |
| P23805 | 0.0385791 | 0.84648543 | NP - P | −0.0602803 | 0.30690166 | 0.84648543 | 0.95218874 |
| A0A6B9SDM5 | 0.03669433 | 0.850233 | NP - P | 0.14483659 | 0.75609923 | 0.850233 | 0.95218874 |
| Q3SYR8 | 0.0329329 | 0.85802324 | NP - P | −0.0878583 | 0.48413635 | 0.85802324 | 0.95218874 |
| G8JKW7 | 0.0328064 | 0.85829304 | NP - P | 0.04051471 | 0.22368325 | 0.85829304 | 0.95218874 |
| A0A6B9SE37 | 0.0327757 | 0.8583586 | NP - P | −0.0415956 | 0.22975858 | 0.8583586 | 0.95218874 |
| A0A3Q1MFI7 | 0.0307389 | 0.86278135 | NP - P | 0.03542728 | 0.20206619 | 0.86278135 | 0.95218874 |
| A6QNW7 | 0.0255283 | 0.87483696 | NP - P | 0.04185659 | 0.2619708 | 0.87483696 | 0.95814036 |
| Q2UVX4 | 0.02457698 | 0.87717075 | NP - P | 0.01443773 | 0.09209471 | 0.87717075 | 0.95814036 |
| Q05443 | 0.021168 | 0.88593909 | NP - P | 0.03787482 | 0.26032193 | 0.88593909 | 0.96278075 |
| A0A3Q1LTF2 | 0.01747575 | 0.89629587 | NP - P | 0.07276112 | 0.55040381 | 0.89629587 | 0.96909147 |
| ENSEMBL:ENSBTAP00000024466 | 0.01472766 | 0.90475234 | NP - P | 0.04122365 | 0.33968752 | 0.90475234 | 0.97329418 |
| Q17QH1 | 0.01100737 | 0.9176027 | NP - P | 0.01718874 | 0.16383325 | 0.9176027 | 0.97534064 |
| V6F7X3 | 0.01060001 | 0.91913599 | NP - P | 0.01993808 | 0.19365563 | 0.91913599 | 0.97534064 |
| A6QM09 | 0.01027213 | 0.92039187 | NP - P | 0.024361 | 0.24036144 | 0.92039187 | 0.97534064 |
| A0A3Q1M616 | 0.00424829 | 0.94875 | NP - P | −0.0123403 | 0.18932937 | 0.94875 | 0.99547226 |
| P00741 | 0.00292417 | 0.95747063 | NP - P | −0.0135305 | 0.25021415 | 0.95747063 | 0.99547226 |
| Q29RU4 | 0.00214263 | 0.96358995 | NP - P | 0.0224144 | 0.48423206 | 0.96358995 | 0.99547226 |
| F1MY85 | 0.0020946 | 0.96400003 | NP - P | −0.0236097 | 0.5158681 | 0.96400003 | 0.99547226 |
| A0A6B9SED3 | 0.00197551 | 0.96503767 | NP - P | −0.0195037 | 0.43881079 | 0.96503767 | 0.99547226 |
| A0A3Q1LK49 | 0.00109262 | 0.97399466 | NP - P | −0.0040958 | 0.12390914 | 0.97399466 | 0.99547226 |
| A0A6B9SDZ9 | 0.00072303 | 0.97884392 | NP - P | −0.0118302 | 0.43996068 | 0.97884392 | 0.99547226 |
| P35908 | 0.00065001 | 0.97994036 | NP - P | −0.0079721 | 0.31268758 | 0.97994036 | 0.99547226 |
| A0A6B9SE04 | 0.00036781 | 0.98490984 | NP - P | 0.01135781 | 0.59222108 | 0.98490984 | 0.99547226 |
| Q5GN72 | 0.00020868 | 0.98863328 | NP - P | −0.0027653 | 0.19142431 | 0.98863328 | 0.99547226 |
| A5D9D2 | 3.87E−05 | 0.99510235 | NP - P | 0.00084126 | 0.13516029 | 0.99510235 | 0.99547226 |
| Q58D62 | 3.31E−05 | 0.99547226 | NP - P | −0.0014651 | 0.25461513 | 0.99547226 | 0.99547226 |

| | Linear Models for Microarray | | | | | |
|---|---|---|---|---|---|---|
| protein | logFC | AveExpr | t | P.Value | adj.P.Val | B |
| A5D798 | 0.58745101 | 32.1157103 | 8.56016667 | 8.68E−11 | 1.85E−08 | 14.4261528 |
| P19034 | −1.3065092 | 24.7957092 | −6.0399132 | 3.36E−07 | 2.52E−05 | 6.24800111 |
| F1MYX5 | −1.0872934 | 25.4121547 | −5.8667858 | 5.98E−07 | 3.18E−05 | 5.69274611 |
| P01035 | −1.1241175 | 25.7240112 | −3.7314546 | 0.00056055 | 0.00746233 | −0.9985323 |
| F1N102 | −0.7284703 | 30.6125888 | −5.7137908 | 9.94E−07 | 4.23E−05 | 5.16951016 |
| P02768 | −0.6415732 | 31.2698849 | −4.6249933 | 3.50E−05 | 0.00120339 | 1.67888277 |
| G3MY71 | 1.00787761 | 27.6991677 | 3.87858769 | 0.00036023 | 0.00548063 | −0.5881268 |
| P00744 | −1.1836715 | 25.4931984 | −4.1141205 | 0.00017519 | 0.00373153 | 0.11118766 |
| A0A6B9SCM2 | −0.872734 | 26.9117075 | −3.187607 | 0.00269428 | 0.02495136 | −2.5089951 |
| Q5E9E3 | −0.4044085 | 29.3256747 | −2.6256016 | 0.0119811 | 0.0510395 | −3.8909885 |
| Q2HJF0 | −0.363425 | 30.115146 | −2.8457204 | 0.00679842 | 0.0362016 | −3.3680487 |
| P80109 | −0.4912293 | 30.3882863 | −3.0427757 | 0.00401327 | 0.02849425 | −2.8760547 |
| Q95121 | −0.3140941 | 30.408229 | −3.2288076 | 0.00240163 | 0.02325218 | −2.4062767 |
| G1K122 | −0.3576473 | 32.8772278 | −4.3691464 | 7.90E−05 | 0.00186968 | 0.88730414 |
| P17690 | −0.253405 | 31.5299459 | −3.1526791 | 0.00296848 | 0.02634529 | −2.5854658 |
| G5E5V0 | 0.39741235 | 30.2134629 | 3.06500697 | 0.00377739 | 0.02849425 | −2.8214001 |
| A0A6B9SE58 | −0.5398681 | 25.5297728 | −3.2470945 | 0.00228163 | 0.02314227 | −2.3113475 |
| Q5EA67 | 0.48559692 | 31.7674102 | 3.04921235 | 0.00394359 | 0.02849425 | −2.8627557 |
| P06868 | −0.5376841 | 32.2829586 | −2.8914788 | 0.00602495 | 0.03468416 | −3.2681525 |
| A0A6B9SEF7 | 0.39685106 | 28.3522751 | 3.05181054 | 0.00391579 | 0.02849425 | −2.8369444 |
| F6QND5 | −0.1516412 | 35.8720878 | −2.8031914 | 0.00759923 | 0.03764268 | −3.4736787 |
| F1N0I3 | 0.47763868 | 27.5610284 | 2.87736731 | 0.00625426 | 0.03505678 | −3.2786096 |
| Q2KIU3 | 0.13929521 | 33.2955001 | 2.72560186 | 0.0092898 | 0.04210056 | −3.6412314 |
| A0A3Q1MI29 | 0.71337104 | 28.2126517 | 4.58714658 | 3.95E−05 | 0.00120339 | 1.59788374 |
| Q2KIX7 | −0.2754314 | 31.1315727 | −3.2737421 | 0.0021169 | 0.02254502 | −2.2728907 |
| E1BF81 | −0.4178106 | 28.7728131 | −2.4599773 | 0.01805036 | 0.06537525 | −4.2418772 |
| G3X6N3 | −0.2939122 | 32.9453184 | −2.9411974 | 0.00527803 | 0.03306531 | −3.0784591 |
| A0A6B9SBF6 | −0.6024592 | 29.4467514 | −2.9676621 | 0.00491668 | 0.03306531 | −3.061755 |
| F1MZ96 | 0.3859242 | 35.3140838 | 3.52060694 | 0.00104355 | 0.01307503 | −1.5897824 |
| A5PJ69 | −0.4713081 | 29.4974928 | −2.8974436 | 0.0059304 | 0.03468416 | −3.215072 |
| A0A3Q1MS75 | −0.5537905 | 27.1148612 | −4.4021835 | 7.12E−05 | 0.00186968 | 1.01493401 |
| B8Y9T0 | −0.5112783 | 32.0459892 | −2.8637627 | 0.00648302 | 0.03540725 | −3.3128392 |
| E1B805 | −0.1811009 | 30.7065157 | −2.4568781 | 0.01818675 | 0.06537525 | −4.2681842 |
| Q2KJH6 | −0.2421576 | 27.9113421 | −2.1244851 | 0.03950126 | 0.11475668 | −4.9359539 |
| A0A3Q1MNN6 | 0.26712 | 32.2129873 | 2.91708318 | 0.00562877 | 0.03425511 | −3.1811117 |
| Q2KIF2 | −0.2499785 | 31.8261094 | −3.9673512 | 0.00027503 | 0.00517965 | −0.3013365 |
| A0A3Q1MGP1 | 0.15498616 | 30.9289848 | 2.23651755 | 0.03063301 | 0.09814707 | −4.6796024 |
| A0A3Q1LI44 | 0.2643051 | 31.7242687 | 2.95421989 | 0.00509722 | 0.03306531 | −3.0630748 |
| P02676 | −0.4188758 | 31.7046529 | −6.0236194 | 3.55E−07 | 2.52E−05 | 6.22456726 |
| ENSEMBL:ENSBTAP00000038329 | −0.3222685 | 28.7515079 | −1.7871828 | 0.08105728 | 0.19735299 | −5.5461121 |
| A0A3Q1LQ21 | −0.5427338 | 29.3039483 | −2.648958 | 0.01129509 | 0.04909907 | −3.8389057 |
| F1MAV0 | 0.192735 | 35.0878808 | 2.80642353 | 0.00753542 | 0.03764268 | −3.4469367 |
| A0A6B9SBI8 | −0.4258463 | 30.0458701 | −2.337287 | 0.0242177 | 0.0818789 | −4.5178302 |
| Q7SIH1 | −0.1130041 | 40.9994292 | −2.272692 | 0.02817392 | 0.09232376 | −4.6465495 |
| Q68RU0 | 0.41096923 | 30.2858579 | 2.43422224 | 0.01921251 | 0.0670863 | −4.2764996 |
| P55906 | −0.811888 | 24.6255144 | −2.1728315 | 0.03542898 | 0.10780534 | −4.8496595 |
| A0A3Q1ML26 | −0.358597 | 30.5956447 | −3.9439811 | 0.00029534 | 0.00517965 | −0.3144227 |
| A6QPQ2 | 0.26152809 | 31.4269694 | 3.34049293 | 0.00175242 | 0.02073696 | −2.0310782 |
| A0A3Q1LI40 | −0.2300521 | 31.6000094 | −2.4517258 | 0.01841556 | 0.06537525 | −4.2743821 |
| A0A140T897 | −0.0591138 | 40.8211474 | −1.6541006 | 0.10549205 | 0.23904049 | −5.734176 |
| A0A6B9SBL4 | −0.767162 | 24.512733 | −2.2988915 | 0.0265045 | 0.08821029 | −4.5765567 |
| A0A3Q1M5R4 | −0.9595038 | 26.6389252 | −1.8227637 | 0.07539511 | 0.18893129 | −5.4840551 |
| A0A6B9SCG2 | −0.857029 | 23.5851006 | −2.7397089 | 0.00895863 | 0.04180639 | −3.5612404 |
| A0A3Q1MIL5 | 0.45205928 | 28.4600181 | 2.35570166 | 0.0231851 | 0.07965202 | −4.4653095 |
| F1MS32 | 0.5523264 | 27.0682694 | 2.55931812 | 0.01414141 | 0.05792538 | −4.009613 |
| A0A6B9SBU4 | −0.2310152 | 30.1305018 | −1.4737204 | 0.14794232 | 0.31830013 | −6.0689623 |
| A0A6B9SDY9 | −0.1787598 | 32.9838136 | −2.4721116 | 0.01752524 | 0.06537525 | −4.1931671 |
| P00761 | −0.55299 | 32.2081182 | −1.7182807 | 0.09304013 | 0.21777526 | −5.6857725 |
| A0A6B9SDP2 | 0.15403008 | 28.0506355 | 1.37885685 | 0.17516946 | 0.35876052 | −6.0972368 |
| K4JB97 | −0.7490032 | 33.2440835 | −3.7598063 | 0.00051503 | 0.0073135 | −0.8781646 |
| A6QP30 | −0.239619 | 28.2948573 | −2.0296339 | 0.04869953 | 0.13648683 | −5.1182017 |
| A0A3Q1NJR8 | −0.2240692 | 35.4733765 | −2.5687675 | 0.01381308 | 0.05768992 | −4.001383 |
| P01888 | 0.32364041 | 28.3782732 | 2.52026022 | 0.01557552 | 0.06143675 | −4.0740267 |
| I7CT57 | −0.1188278 | 36.4159344 | −1.9862347 | 0.05349571 | 0.14608444 | −5.19704 |
| Q3MHH8 | 0.7456537 | 28.9712345 | 3.10252681 | 0.00340863 | 0.02792455 | −2.626228 |
| G3N3S2 | −0.3233423 | 29.9488212 | −2.2331335 | 0.03087255 | 0.09814707 | −4.7294594 |
| Q28085 | −0.2589407 | 32.4033312 | −2.8110015 | 0.00744589 | 0.03764268 | −3.4373177 |
| Q29437 | −0.1157645 | 34.7913965 | −1.7420401 | 0.08875147 | 0.21240521 | −5.656735 |
| P34955 | −0.1329231 | 35.3238228 | −2.0401359 | 0.04759669 | 0.13517461 | −5.0857797 |
| A0A6B9SCC1 | 0.45132733 | 30.2524818 | 2.49239658 | 0.01667827 | 0.0645904 | −4.1572392 |
| A0A6B9SBM2 | −0.3133683 | 28.5574426 | −2.2158673 | 0.03212081 | 0.09915556 | −4.7345948 |
| A0A6B9SDM9 | 0.52693658 | 26.1433437 | 1.9956086 | 0.0524265 | 0.14502394 | −5.1921636 |
| A0A3Q1NEQ0 | −0.2073302 | 31.767644 | −1.8678823 | 0.06869933 | 0.17420186 | −5.4253009 |
| F6R7W8 | 0.32643254 | 28.3909885 | 3.13601489 | 0.00310841 | 0.02648363 | −2.5940936 |
| A0A6B9SBT1 | 0.25454787 | 30.7417558 | 1.04928505 | 0.29999068 | 0.49920324 | −6.5476463 |
| P41361 | −0.1321301 | 32.4057381 | −1.0990197 | 0.27796036 | 0.48529144 | −6.5355116 |
| Q29443 | 0.18147702 | 35.4798267 | 1.19279437 | 0.2395817 | 0.45295321 | −6.4249638 |
| P13645 | −0.1792335 | 29.29034 | −1.2237783 | 0.2277925 | 0.44108911 | −6.3497681 |
| G3N0V0 | 0.16831372 | 38.1353331 | 1.6437767 | 0.10761756 | 0.24128991 | −5.7916206 |
| Q28107 | 0.1170496 | 32.2176925 | 1.41335462 | 0.16485068 | 0.34765539 | −6.1329473 |
| G3MYZ3 | −0.361605 | 28.9179066 | −2.1286342 | 0.0391363 | 0.11475668 | −4.9431909 |
| A0A6B9SBJ6 | 0.37934824 | 28.602501 | 2.12034382 | 0.03986852 | 0.11475668 | −4.9339159 |
| ENSEMBL:ENSBTAP00000014147 | 0.14116144 | 32.4698836 | 1.67756363 | 0.10078953 | 0.23334966 | −5.7296013 |
| ENSEMBL:ENSBTAP00000031900 | −0.1050592 | 34.7908957 | −1.9799558 | 0.0542224 | 0.14619456 | −5.2039717 |
| B0JYQ0 | −0.2045027 | 34.9014324 | −2.6905365 | 0.01016255 | 0.04509629 | −3.7262591 |
| A0A3Q1LKB0 | 0.46614725 | 27.0455629 | 3.29106774 | 0.00201593 | 0.02254502 | −2.1977103 |
| F1MLW8 | 0.25595156 | 31.235079 | 1.13683754 | 0.26198992 | 0.47695601 | −6.4841167 |
| A0A6B9SCN1 | 0.32461345 | 30.0590686 | 2.94383389 | 0.00524095 | 0.03306531 | −3.101225 |
| A0A0A0MP92 | −0.180952 | 34.5978364 | −2.7366882 | 0.00902861 | 0.04180639 | −3.5853087 |
| A6QPP2 | −0.2444909 | 32.8678981 | −2.4730578 | 0.01748488 | 0.06537525 | −4.1932345 |
| Q1RMH5 | −0.3324943 | 26.1319952 | −1.3414684 | 0.186909 | 0.37915826 | −6.2359435 |
| A0A3Q1LJT1 | 0.76155307 | 29.3463885 | 3.92163255 | 0.00031613 | 0.00517965 | −0.4452321 |
| A0A3Q1N1A7 | 0.1423997 | 28.6667387 | 1.28369553 | 0.20621914 | 0.41438374 | −6.2883048 |
| A0A6B9SBI1 | −0.3077746 | 28.5568434 | −0.8915559 | 0.37766229 | 0.57871991 | −6.7285414 |
| A0A6B9SBJ3 | −0.3066 | 27.4589574 | −2.5434428 | 0.0147091 | 0.05911394 | −4.0508293 |
| A2I7N0 | −0.1821581 | 34.8942015 | −1.5169738 | 0.13669376 | 0.30328928 | −5.9927751 |
| P17697 | −0.1060203 | 32.1998716 | −1.1820772 | 0.24376154 | 0.4554492 | −6.3878617 |
| Q3SYR0 | 0.15734986 | 29.8530383 | 1.72530614 | 0.09175443 | 0.21715215 | −5.6458768 |
| A0A6B9SDW4 | 0.33544239 | 29.26833 | 1.9071137 | 0.06329517 | 0.16675734 | −5.3178173 |
| Q3SZV7 | −0.0857662 | 35.8619488 | −1.1242909 | 0.26721396 | 0.47829054 | −6.510121 |
| A5PK72 | −0.3728877 | 28.9702928 | −0.9560611 | 0.34446046 | 0.54823403 | −6.6781854 |
| A0A3Q1M3L6 | 0.03213546 | 37.3834289 | 0.25873528 | 0.79709134 | 0.89358135 | −7.0625147 |
| P01045-2 | −0.2145135 | 30.0730341 | −1.2257591 | 0.2270536 | 0.44108911 | −6.3310948 |
| Q9TTE1 | −0.1916987 | 36.020943 | −1.906215 | 0.06341476 | 0.16675734 | −5.343066 |
| P35527 | −0.2223865 | 33.0254441 | −0.9312395 | 0.35700154 | 0.56163377 | −6.6724601 |
| F1MKS5 | −0.2787232 | 31.28514 | −1.0841641 | 0.28441802 | 0.48700665 | −6.5577011 |
| A0A6B9SBR2 | −0.3393687 | 26.9008636 | −1.2495901 | 0.21830268 | 0.4342823 | −6.3313 |
| E1BCW0 | −0.4429618 | 27.0225012 | −2.2162415 | 0.03209329 | 0.09915556 | −4.7583019 |
| F1MJ12 | 0.23080594 | 28.2796475 | 0.83264861 | 0.40970815 | 0.59957745 | −6.7782121 |
| Q95122 | −0.2226944 | 26.1438528 | −0.7765281 | 0.44174739 | 0.62530665 | −6.8273306 |
| Q2KIT0 | −0.1271797 | 32.226132 | −1.8879249 | 0.06589125 | 0.17115654 | −5.3669402 |
| P02672 | 0.12236395 | 32.5662369 | 0.95518443 | 0.3448984 | 0.54823403 | −6.6533046 |
| ENSEMBL:ENSBTAP00000016046 | 0.16225317 | 28.7891286 | 0.77573624 | 0.44220987 | 0.62530665 | −6.8259283 |
| A0A6B9SBM1 | 0.06780296 | 28.2298794 | 0.26828222 | 0.78978295 | 0.89198872 | −7.0712637 |
| A0A3Q1NJB1 | −0.0757459 | 33.5913827 | −0.8851854 | 0.38104877 | 0.57973849 | −6.7421324 |
| P28800 | −0.0559323 | 33.2402296 | −0.7369361 | 0.46521926 | 0.63115734 | −6.8607553 |
| Q32PA1 | −0.0886564 | 27.6640713 | −0.352811 | 0.72597792 | 0.84039836 | −7.0458703 |
| E1B7G5 | −0.2087282 | 28.4766749 | −1.3890456 | 0.17207118 | 0.35876052 | −6.1755047 |
| E1BH06 | 0.19101828 | 34.383378 | 2.15283066 | 0.03706644 | 0.11119932 | −4.8393696 |
| A0A6B9SCH7 | −0.1022087 | 26.2085528 | −0.3566913 | 0.72309176 | 0.84039836 | −7.0572293 |
| A0A6B9SCJ6 | 0.12961445 | 26.9876649 | 0.42192758 | 0.6752094 | 0.7963239 | −7.0349767 |
| A0A6B9SE69 | −0.1507626 | 28.3668078 | −1.0764028 | 0.28783329 | 0.48700665 | −6.5291104 |
| A0A0A0MPA0 | −0.4322443 | 32.2519274 | −0.990626 | 0.32748758 | 0.5324798 | −6.6439806 |
| Q17QC8 | −0.2417623 | 26.8745117 | −1.8736726 | 0.0678778 | 0.17419243 | −5.3970092 |
| Q32L76 | −0.331206 | 29.2475234 | −0.9229825 | 0.36123862 | 0.56163377 | −6.6790311 |
| A0A6B9SE85 | −0.3612794 | 28.7381476 | −1.5111796 | 0.13815975 | 0.30338173 | −5.9839325 |
| K4JDT2 | 0.04376319 | 34.8763146 | 0.26664316 | 0.79103635 | 0.89198872 | −7.0529444 |
| Q2KI67 | 0.18493092 | 25.916793 | 0.65049571 | 0.51888129 | 0.69076072 | −6.9245818 |
| G3N1H5 | −0.1895271 | 27.8630102 | −0.6018532 | 0.55047645 | 0.71061506 | −6.872525 |
| E1BH94 | −0.421593 | 33.1343013 | −1.7842745 | 0.08153551 | 0.19735299 | −5.5426337 |
| A5PKC2 | 0.1368836 | 29.0069495 | 0.82481258 | 0.41409392 | 0.60001364 | −6.7907365 |
| A0A6B9SDY7 | 0.12758904 | 32.2337198 | 1.10834006 | 0.27396198 | 0.48529144 | −6.5229109 |
| A0A6B9SBR8 | −0.2328042 | 27.9910689 | −1.0134073 | 0.31661346 | 0.51875898 | −6.5390471 |
| C4T8B4 | −0.1318558 | 28.690587 | −0.8940934 | 0.37631877 | 0.57871991 | −6.7040116 |
| P01030 | 0.24969521 | 29.1478012 | 0.84744848 | 0.40150318 | 0.59957745 | −6.7840101 |
| A0A6B9SDY5 | −0.140452 | 29.4906837 | −0.7502884 | 0.45722416 | 0.62530665 | −6.8454303 |
| Q9TS85 | 0.05671081 | 31.6240684 | 0.47783776 | 0.63521983 | 0.77315328 | −6.9387571 |
| A0A3Q1LPG0 | −0.2317194 | 35.0256469 | −1.7956893 | 0.07967205 | 0.19732728 | −5.4767329 |
| Q3T004 | 0.42258887 | 28.065455 | 1.67148787 | 0.10199034 | 0.23359077 | −5.7468119 |
| O02659 | 0.16712097 | 29.0770565 | 0.7298516 | 0.4694938 | 0.63292519 | −6.8604371 |
| G3N0S9 | 0.07241431 | 32.8965221 | 0.7112997 | 0.48079328 | 0.64408156 | −6.8668812 |
| A0A452DHX8 | −0.044595 | 27.6942591 | −0.2766994 | 0.78335528 | 0.89198872 | −7.0773803 |
| A0A3Q1MIN7 | 0.16617372 | 27.8206208 | 0.84538823 | 0.40263922 | 0.59957745 | −6.7473343 |
| V6F9A3 | −0.1177065 | 30.0832961 | −0.7490353 | 0.45797106 | 0.62530665 | −6.8518075 |
| A0A6B9SE14 | −0.0353293 | 28.3190963 | −0.177886 | 0.85965956 | 0.92013812 | −7.0905861 |
| Q58DL9 | −0.2360556 | 26.0409859 | −0.7676374 | 0.44695627 | 0.62530665 | −6.8351467 |
| A5D7Q2 | −0.3785168 | 30.1320961 | −1.1909453 | 0.24029912 | 0.45295321 | −6.4000157 |
| A0A3Q1MJT2 | −0.0802668 | 35.6456654 | −0.9234688 | 0.36098813 | 0.56163377 | −6.6641802 |
| G5E5A8 | 0.2441961 | 27.4960098 | 1.03546528 | 0.3063209 | 0.50578567 | −6.5696156 |
| A0A6B9SF17 | 0.15464974 | 31.0369404 | 0.420294 | 0.67639295 | 0.7963239 | −7.0126271 |
| P15497 | 0.04634266 | 38.6272834 | 0.64399336 | 0.523048 | 0.69198276 | −6.9264128 |
| Q3T101 | 0.21258115 | 32.1091066 | 1.48615017 | 0.1446372 | 0.31436452 | −5.9728226 |
| A5PK49 | −0.2608182 | 29.8585875 | −1.1280851 | 0.26562641 | 0.47829054 | −6.4432461 |
| F1MMK9 | 0.11583211 | 30.7028847 | 1.16163488 | 0.25188091 | 0.46652725 | −6.3297513 |
| A0A6B9SDR8 | 0.09459846 | 29.5951882 | 0.75956344 | 0.45171779 | 0.62530665 | −6.7815941 |
| P04264 | 0.13597716 | 31.3994259 | 1.08738446 | 0.28300927 | 0.48700665 | −6.4703144 |
| Q2KIS7 | 0.00617267 | 33.0357882 | 0.10084533 | 0.92014856 | 0.94779149 | −7.1156213 |
| A0A452DK44 | 0.04027951 | 28.6690698 | 0.18143488 | 0.85689074 | 0.92013812 | −7.095552 |
| A0A452DHZ3 | 0.01050807 | 28.2366426 | 0.14045187 | 0.88896804 | 0.93873379 | −7.0984061 |
| A0A452DJ14 | 0.06860953 | 28.7025459 | 0.75799469 | 0.45264638 | 0.62530665 | −6.8368321 |
| E1BMJ0 | −0.1058204 | 28.26015 | −1.1425443 | 0.25963812 | 0.47674931 | −6.4238273 |
| A0A6B9SC93 | −0.6254885 | 27.361465 | −2.7571422 | 0.00856447 | 0.04145983 | −3.5073738 |
| A0A452DI66 | −0.0649437 | 34.2547907 | −0.8043644 | 0.42567338 | 0.61262453 | −6.8095467 |
| A0A6B9SDW5 | −0.1636723 | 29.5833802 | −1.0996702 | 0.27767995 | 0.48529144 | −6.4895074 |
| E1B726 | −0.0346701 | 34.1857691 | −0.4516547 | 0.65381949 | 0.7823795 | −7.0450678 |
| Q3SZZ9 | −0.0125109 | 35.872499 | −0.2093689 | 0.83516288 | 0.90763312 | −7.1101117 |
| P12763 | −0.1358408 | 33.5047854 | −0.9644908 | 0.34026841 | 0.54823403 | −6.6522345 |
| Q3MHN2 | −0.0915051 | 30.1053313 | −0.8463313 | 0.40211893 | 0.59957745 | −6.7079391 |
| ENSEMBL:ENSBTAP00000007350 | −0.2170139 | 33.1784788 | −1.4205966 | 0.16274591 | 0.34664878 | −6.0996745 |
| A0A3B0J3V0 | −0.0346551 | 33.8382792 | −0.3806835 | 0.70533865 | 0.82547875 | −6.9792206 |
| D4QBB4 | 0.03964426 | 34.7084249 | 0.11741664 | 0.9070843 | 0.94409358 | −7.1333151 |
| Q1RMN8 | −0.1886016 | 34.2637312 | −0.6147442 | 0.54200855 | 0.70395012 | −6.939225 |
| Q2KJ63 | 0.10881643 | 28.3527931 | 1.06110572 | 0.29464831 | 0.49417394 | −6.5548953 |
| P60712 | 0.03951982 | 27.3848134 | 0.22370167 | 0.82406373 | 0.90763312 | −7.089103 |
| A0A452DIP8 | 0.1240312 | 28.0216455 | 0.62718984 | 0.53389753 | 0.70197638 | −6.8969211 |
| A0A6B9SE50 | −0.1604523 | 27.8888996 | −0.5163645 | 0.60828238 | 0.75327993 | −7.0024041 |
| Q5EAD3 | 0.12552284 | 27.6034674 | 1.38241978 | 0.17408114 | 0.35876052 | −6.1286378 |
| A0A6B9SBG6 | 0.09831446 | 26.549634 | 0.5819736 | 0.56366598 | 0.71695998 | −6.9221007 |
| O46375 | 0.04149664 | 34.2782903 | 0.55617931 | 0.58101152 | 0.73228079 | −6.952378 |
| A0A6B9SBF3 | −0.3711439 | 29.0997679 | −1.0758256 | 0.28808844 | 0.48700665 | −6.5174832 |
| A0A3Q1ME55 | 0.12500048 | 28.375069 | 0.83370672 | 0.40911813 | 0.59957745 | −6.7414413 |
| G5E5T5 | 0.10893892 | 32.3252819 | 0.46720879 | 0.6427425 | 0.7778645 | −7.0320342 |
| A0A3Q1M5Q6 | −0.0885551 | 31.0059719 | −0.4516689 | 0.6538093 | 0.7823795 | −7.0130952 |
| G3N1U4 | −0.0587254 | 33.8060804 | −0.5792435 | 0.56548956 | 0.71695998 | −6.923241 |
| F1MDS0 | −0.0598071 | 24.9169588 | −0.1388136 | 0.89025458 | 0.93873379 | −7.1283554 |
| P01017 | −0.1466645 | 32.8904557 | −1.1948197 | 0.23879771 | 0.45295321 | −6.4057709 |
| Q09TE3 | 0.01312161 | 31.2295668 | 0.11544845 | 0.90863466 | 0.94409358 | −7.1158694 |
| P23805 | −0.0121946 | 30.3358743 | −0.0620711 | 0.95079787 | 0.96899496 | −7.1158039 |
| A0A6B9SDM5 | −0.2168912 | 23.7108932 | −0.4198864 | 0.67668838 | 0.7963239 | −7.0319521 |
| Q3SYR8 | 0.04710605 | 28.8194158 | 0.12116722 | 0.90413094 | 0.94409358 | −7.1409997 |
| G8JKW7 | −0.0726747 | 33.2961156 | −0.50841 | 0.61380082 | 0.75572009 | −6.9931025 |
| A0A6B9SE37 | 0.04726409 | 28.4424911 | 0.26605932 | 0.79148295 | 0.89198872 | −7.1023045 |
| A0A3Q1MFI7 | −0.035666 | 30.5980522 | −0.2122361 | 0.83293978 | 0.90763312 | −7.1186159 |
| A6QNW7 | 0.11516524 | 32.694148 | 1.24436527 | 0.22019948 | 0.4342823 | −6.2597387 |
| Q2UVX4 | −0.0548156 | 38.5074236 | −0.8303746 | 0.41097797 | 0.59957745 | −6.7766219 |
| Q05443 | −0.0918959 | 28.3576874 | −0.544187 | 0.58916306 | 0.73818666 | −6.979035 |
| A0A3Q1LTF2 | 0.00974932 | 26.712127 | 0.03930722 | 0.96882993 | 0.97339989 | −7.1060862 |
| ENSEMBL:ENSBTAP00000024466 | 0.11388003 | 33.8667287 | 0.77306702 | 0.44377088 | 0.62530665 | −6.8076332 |
| Q17QH1 | −0.0373142 | 27.793988 | −0.2860152 | 0.77625918 | 0.89198872 | −7.0770437 |
| V6F7X3 | 0.00543479 | 32.5653286 | 0.04414977 | 0.96499227 | 0.97339989 | −7.1231961 |
| A6QM09 | 0.01730978 | 38.4098165 | 0.09964834 | 0.92109314 | 0.94779149 | −7.1247295 |
| A0A3Q1M616 | 0.07971766 | 26.851119 | 0.52932858 | 0.59933791 | 0.74654372 | −6.9744958 |
| P00741 | 0.09706329 | 29.9024016 | 0.62052043 | 0.53823619 | 0.70333932 | −6.9336012 |
| Q29RU4 | −0.0509607 | 30.8210763 | −0.2275524 | 0.82108783 | 0.90763312 | −7.04009 |
| F1MY85 | −0.0267989 | 34.5419772 | −0.0533821 | 0.95767816 | 0.97135927 | −7.1450567 |
| A0A6B9SED3 | −0.0383505 | 26.9092486 | −0.1413603 | 0.8882548 | 0.93873379 | −7.0996166 |
| A0A3Q1LK49 | −0.0026649 | 35.9590715 | −0.029336 | 0.97673425 | 0.97673425 | −7.1319788 |
| A0A6B9SDZ9 | −0.0201121 | 30.0045755 | −0.0630018 | 0.9500611 | 0.96899496 | −7.1406041 |
| P35908 | 0.03066031 | 27.6551718 | 0.20933019 | 0.83519292 | 0.90763312 | −7.0662771 |
| A0A6B9SE04 | 0.18642346 | 27.7235504 | 0.58331083 | 0.56277385 | 0.71695998 | −6.9319905 |
| Q5GN72 | −0.0242342 | 32.0333933 | −0.2005153 | 0.84203624 | 0.91042497 | −7.1035752 |
| A5D9D2 | −0.0461093 | 34.6246426 | −0.4973485 | 0.62151249 | 0.76081702 | −7.0080089 |
| Q58D62 | 0.04089134 | 30.4006319 | 0.24816953 | 0.80520115 | 0.89794684 | −7.1022728 |

Integrative Multi-Omics Analysis Identifies Molecular Features that Classify Heifers Based on their Fertility Potential

When each data were evaluated independently, the quantification of 22 and 23 gene and protein relative abundances accounted for 44.1% and 16.6% of the variance associated with fertility classification, respectively, and the genotypic information of 59 SNPs explained 70.1% of the variance associated with fertility classification. Overall, there were four factors identified in the analysis with the potential to distinguish the samples based on their fertility status, out of which three were most representative with Factors one, two, and three being mostly dominated by genotype, transcript, and protein data, respectively (FIG. 4A). Factors one, two, and three separated most of the samples based on their fertility classification except two, three, and five samples, respectively (FIG. 4B).

Notably, the top nine SNPs that explained most of the variance related to Factor one are located in a window on chromosome 5 spanning from nucleotide 118332762 to 118345383. The tenth SNP was the top significant polymorphism identified on chromosome 12 nucleotide 85648422 according to our Fisher's exact test contrasting heifers of different fertility potential (FIG. 4C). Among the genes whose transcript abundance explained the variance related to Factor two, Applicant identified the following annotated genes: ArfGAP with SH3 domain, ankyrin repeat and PH domain 3 (ASAP3), ATP synthase membrane subunit c locus 1 (ATP5MC1), Centrosomal protein 170 (CEP170), Myeloid derived growth factor (MYDGF), Coiled-coil domain containing 34 (CCDC34), RAD51 associated protein 1 (RAD51AP1), and Ubiquinol-cytochrome c reductase complex III subunit VII (UQCRQ) (FIG. 4C). Among the proteins whose abundance explained the variance related to Factor three, the following were annotated to known genes: Apolipoprotein C-II (APOC2), Lymphocyte cytosolic protein 1 (LCP1), Vitamin K-dependent protein Z (PROZ), Albumin (ALB), Serotransferrin-like (LOC525947), Complement component C8 beta chain (C8B), Pigment epithelium-derived factor (SERPINF1), Phosphatidylinositol-glycan-specific phospholipase D (GPLD1), Alpha-ketoglutarate-dependent dioxygenase FTO (FTO) (FIG. 4C). Collectively, data from the genotypes, transcriptome, and proteome clustered 21 out of 22 heifers correctly based on their fertility status, with only one Fertile heifer clustering with the group of Sub-Fertile heifers.

### Discussion

Reproduction is a multidimensional biological function in mammals that can be partitioned into multiple components or traits⁸⁵, and as a consequence, infertility is a complex phenotype with multifactorial origins, including a strong genetic component^{53,54}. This work at least addressed two critical questions regarding the underlying biology of infertility: (a) whether multiple layers of molecular information, present in the circulatory system, would differ based on female fertility fitness; and (b) whether the integrative analysis of multiple layers of molecular information would be a better predictor of the causes of infertility. Our analysis identified molecular signatures in the genome, transcriptome, and proteome that provide important insights about the root causes of infertility.

Neither one of the significant SNPs were located in a region previously associated with female reproductive traits⁸⁶. These SNPs have also not been previously reported to be associated with fertility traits in previous investigations that focused on sire-centric models^{4,33,34,35,36,37}, nor on studies that focused on genotyped heifers only^{32,38}. However, it is notable that the polymorphism rs110918927 is in the gene EML6, which produces a protein that participates in the function of spindle microtubules in oocytes⁸⁷. Knockdown of this protein in mice oocytes at the germinal vesicle stage impairs spindle morphology and increases aneuploidy⁸⁷ in oocytes that progress to the metaphase II stage in the absence of EML6⁸⁸. The gene EML6 also produces transcripts in bovine oocytes⁸⁹, and the significant SNP in this gene is a strong indication of a functional connection to reduced oocyte developmental competence in the Sub-Fertile group of heifers.

Genes differentially expressed in the peripheral white blood cells have been associated with fertility in heifers^{39,40,41}. The protein APMAP exhibits arylesterase activity, which is known to protect lipoproteins from oxidation⁹⁰. Importantly, the APMAP protein regulates adipose composition and metabolic health, and the disruption of the APMAP gene in mice leads to an increase in visceral adipose tissue expansion⁹¹. This protein was also shown to be less abundant in the omental tissue of women diagnosed with polycystic ovary syndrome⁹². Therefore, lower expression of APMAP in the peripheral white blood cells of Sub-Fertile heifers is possibly connected with a metabolic, hormonal or inflammatory disorder that disrupts fertility in heifers.

The Protein DNAI7 composes the axonemal dynein complex and participates in beta-tubulin binding activity and microtubule binding activity, and thus contributes to ciliary beating⁹³. Variants that impair the function of DNAI7 are associated with Primary Ciliary Dyskinesia, with one potential consequence being the abnormal function of cilia and possible impaired transport of the cleaving embryos into the uterus⁹⁴. DNAI7 can also function as a cell cycle regulator, and dysregulated transcript abundance of DNAI7 was associated with nasopharyngeal neoplasm in mice⁹⁵ and lung adenocarcinoma in humans⁹⁶. Since Sub-Fertile heifers have greater abundance of DNAI7 transcripts in their circulating white blood cells, it is possible that dysregulation in the cell cycle has a biological link with subfertility. Further research is required, however, to evaluate whether a dysregulation in the cell cycle linked to upregulation of DNAI7 is connected with increased inflammation⁹¹ associated with less transcripts from APMAP.

The protein Alpha-ketoglutarate-dependent dioxygenase FTO has oxidative demethylation activity of abundant N6-methyladenosine (m⁶A) residues in RNA⁹⁷. The protein FTO preferentially demethylates N6,2′-O-dimethyladenosine (m⁶Am) rather than m⁶A and contributes to a reduced stability of m⁶Am mRNAs⁹⁸. On a systemic level, genomic variants in FTO were associated with symptoms of metabolic disorders⁹⁹, although the effects observed in humans, such elevated body mass indexl^{100,101}, and mice¹⁰² may be contradictory. Also worth noting, a variant on the FTO gene was associated with polycystic ovary syndrome¹⁰³. Interestingly, in mice, the FTO gene is downregulated due to a deficiency in essential amino-acids¹⁰⁴, and deficiency in the FTO protein causes postnatal growth retardation and a significant reduction in adipose tissue and lean body mass¹⁰⁵. Our observation of the FTO abundance in heifers of different fertility potential is an indication that Sub-Fertile heifers could be experiencing a metabolic imbalance, contributing to their lower fertility. Applicant notes that the heifers utilized in this experiment were not nutritionally challenged and thus, our observations are a consequence of their intrinsic biological system and how it may utilize nutrients.

The next step was to interrogate the data Applicant produced in a comprehensive manner. Interestingly, the largest source of variability was observed in the genomic data. Nine of the top ten SNPs that were assigned to Factor one were located in an intron of the TAFA chemokine-like family member 5 (TAFA5) gene. These SNPs are within a quantitative trait loci for milk yield¹⁰⁶, a trait negatively correlated with reproductive traits¹⁰⁷, however, no relationship between genetic variants in this gene and female fertility has been reported previously. None of the top ten genes with transcript abundance relevant for the modeling of the variance were identified as differentially expressed when analyzed independently. This result is not surprising because the identification of significant features using standard statistical approaches for association analysis is not necessarily the best approach for identifying predictive genes associated with complex traits^{108,109}. It was surprising that three out of nine annotated proteins, which composed the top ten proteins that explained most of the variance in factor three, were also identified in our analyses using general linear mixed models. The most interesting result, however, was that all three data modalities were able to separate 21 out of 22 heifers correctly based on their fertility potential. Our results show that molecular differences have strong signals linked to fertility fitness that surpasses their differing genetic background.

### Conclusion

Our interrogation of multiple levels of biological information (genome, transcriptome, and proteome) at a systemic level in heifers highlighted the molecular complexity of female fertility. While the genomic data pointed to a disruption of oocyte developmental competence, the transcriptome and proteomic data point to metabolic dysregulation contributing to subfertility or infertility. Although the differences in molecular profiles identified in our study need to be further validated by mechanistic studies, our results, supported by the current literature, highlight differences in the molecular profile associated with female fertility that transcend the constraints of breed-specific genetic background.

### Data Availability

The transcriptome and proteome data generated and analyzed during the current study are available in the Gene Expression Omnibus and ProteomeXchange repositories under the following identifiers: GSE220220 and PXD038756, respectively. The genotypic data are available from the corresponding author upon reasonable request.

### REFERENCES FOR EXAMPLE 1

1. Henchion, M., Hayes, M., Mullen, A. M., Fenelon, M. & Tiwari, B. Future protein supply and demand: Strategies and factors influencing a sustainable equilibrium. Foods 6(7), 53 (2017).
2. Davis, T. C. & White, R. R. Breeding animals to feed people: The many roles of animal reproduction in ensuring global food security. Theriogenology 150, 27-33 (2020).
3. Moorey, S. E. & Biase, F. H. Beef heifer fertility: Importance of management practices and technological advancements. J Anim. Sci. Biotechnol. 11, 97 (2020).
4. Galliou, J. M. et al. Identification of loci and pathways associated with heifer conception rate in U.S. Holsteins. Genes (Basel) 11(7), 767 (2020).
5. Cushman, R. A., Kill, L. K., Funston, R. N., Mousel, E. M. & Perry, G. A. Heifer calving date positively influences calf weaning weights through six parturitions. J. Anim. Sci. 91(9), 4486-4491 (2013).
6. Lesmeister, J. L., Burfening, P. J. & Blackwell, R. L. Date of first calving in beef cows and subsequent calf production. J. Anim. Sci. 36(1), 1-6 (1973).
7. Damiran, D., Larson, K. A., Pearce, L. T., Erickson, N. E. & Lardner, B. H. A. Effect of calving period on beef cow longevity and lifetime productivity in western Canada. Transl. Anim. Sci. 2(suppl_1), S61-S65 (2018).
8. Boulton, A. C., Rushton, J. & Wathes, D. C. A study of dairy heifer rearing practices from birth to weaning and their associated costs on UK dairy farms. Open J. Anim. Sci. 5, 185-197 (2015).
9. Hoffman, P. C. Optimum body size of Holstein replacement heifers. J. Anim. Sci. 75(3), 836-845 (1997).
10. Heinrichs, A. J. Raising dairy replacements to meet the needs of the twenty-first century. J. Dairy Sci. 76(10), 3179-3187 (1993).
11. Bormann, J. M., Totir, L. R., Kachman, S. D., Fernando, R. L. & Wilson, D. E. Pregnancy rate and first-service conception rate in Angus heifers. J. Anim. Sci. 84, 2022-2025 (2006).
12. Peters, S. O. et al. Bayesian genome-wide association analysis of growth and yearling ultrasound measures of carcass traits in Brangus heifers. J. Anim. Sci. 90(10), 3398-3409 (2012).
13. Fortes, M. R. S. et al. Gene network analyses of first service conception in Brangus heifers: Use of genome and trait associations, hypothalamic-transcriptome information, and transcription factors. J. Anim. Sci. 90, 2894-2906 (2012).
14. Doyle, S. P., Golden, B. L., Green, R. D. & Brinks, J. S. Additive genetic parameter estimates for heifer pregnancy and subsequent reproduction in Angus females. J. Anim. Sci. 78(8), 2091-2098 (2000).
15. Toghiani, S. et al. Genomic prediction of continuous and binary fertility traits of females in a composite beef cattle breed. J. Anim. Sci. 95(11), 4787-4795 (2017).
16. McAllister, C. M., Speidel, S. E., Crews, D. H. Jr. & Enns, R. M. Genetic parameters for intramuscular fat percentage, marbling score, scrotal circumference, and heifer pregnancy in Red Angus cattle. J. Anim. Sci. 89(7), 2068-2072 (2011).
17. Boddhireddy, P. et al. Genomic predictions in Angus cattle: Comparisons of sample size, response variables, and clustering methods for cross-validation. J. Anim. Sci. 92(2), 485-497 (2014).
18. Raheja, K. L., Burnside, E. B. & Schaeffer, L. R. Heifer fertility and its relationship with cow fertility and production traits in Holstein dairy cattle. J. Dairy Sci. 72(10), 2665-2669 (1989).
19. Muir, B. L., Fatehi, J. & Schaeffer, L. R. Genetic relationships between persistency and reproductive performance in first-lactation Canadian Holsteins. J. Dairy Sci. 87(9), 3029-3037 (2004).
20. Walsh, S. W., Williams, E. J. & Evans, A. C. A review of the causes of poor fertility in high milk producing dairy cows. Anim. Reprod Sci. 123(3-4), 127-138 (2011).
21. Kuhn, M. T., Hutchison, J. L. & Wiggans, G. R. Characterization of Holstein heifer fertility in the United States. J. Dairy Sci. 89(12), 4907-4920 (2006).
22. Tiezzi, F., Maltecca, C., Cecchinato, A., Penasa, M. & Bittante, G. Genetic parameters for fertility of dairy heifers and cows at different parities and relationships with production traits in first lactation. J. Dairy Sci. 95(12), 7355-7362 (2012).
23. Jagusiak, W. Fertility measures in Polish Black-and-White cattle. 1. Genetic parameters of heifer fertility traits. J. Anim. Feed Sci. 14(3), 423-433 (2005).
24. Koltes, J. E. et al. A vision for development and utilization of high-throughput phenotyping and big data analytics in livestock. Front. Genet. 10, 1197 (2019).
25. Neupane, M. et al. Loci and pathways associated with uterine capacity for pregnancy and fertility in beef cattle. PLoS ONE 12(12), e0188997 (2017).
26. McDaneld, T. G. et al. Genomewide association study of reproductive efficiency in female cattle. J. Anim. Sci. 92, 1945-1957 (2014).
27. McDaneld, T. G. et al. Y are you not pregnant: Identification of Y chromosome segments in female cattle with decreased repro-ductive efficiency. J. Anim. Sci. 90, 2142-2151 (2012).
28. de Camargo, G. M. et al. Association between JY-1 gene polymorphisms and reproductive traits in beef cattle. Gene 533(2), 477-480 (2014).
29. Dias, M. M. et al. Study of lipid metabolism-related genes as candidate genes of sexual precocity in Nellore cattle. Genet. Mol. Res. 14(1), 234-243 (2015).
30. Irano, N. et al. Genome-wide association study for indicator traits of sexual precocity in Nellore cattle. PLoS ONE 11(8), e0159502 (2016).
31. Junior, G. A. O. et al. Genomic study and medical subject headings enrichment analysis of early pregnancy rate and antral follicle numbers in Nelore heifers. J. Anim. Sci. 95(11), 4796-4812 (2017).
32. Stegemiller, M. R. et al. Genome-wide association analyses of fertility traits in beef heifers. Genes (Basel) 12(2), 217 (2021).
33. Frischknecht, M. et al. Genome-wide association studies of fertility and calving traits in Brown Swiss cattle using imputed whole-genome sequences. BMC Genomics 18(1), 910 (2017).
34. Jiang, J. et al. A Large-scale genome-wide association study in U.S. Holstein cattle. Front. Genet. 10, 412 (2019).
35. Nayeri, S. et al. Genome-wide association for milk production and female fertility traits in Canadian dairy Holstein cattle. BMC Genet. 17(1), 75 (2016).
36. Chen, S. Y. et al. Identifying pleiotropic variants and candidate genes for fertility and reproduction traits in Holstein cattle via association studies based on imputed whole-genome sequence genotypes. BMC Genomics 23(1), 331 (2022).
37. Kiser, J. N. et al. Validation of 46 loci associated with female fertility traits in cattle. BMC Genomics 20(1), 576 (2019).
38. Kiser, J. N. et al. Identification of loci associated with conception rate in primiparous Holstein cows. BMC Genomics 20(1), 840 (2019).
39. Moorey, S. E. et al. Rewiring of gene expression in circulating white blood cells is associated with pregnancy outcome in heifers (Bos taurus). Sci. Rep. 10(1), 16786 (2020).
40. Dickinson, S. E. et al. Transcriptome profiles in peripheral white blood cells at the time of artificial insemination discriminate beef heifers with different fertility potential. BMC Genomics https://doi.org/10.1186/s12864-018-4505-4 (2018).
41. Dickinson, S. E. & Biase, F. H. Transcriptome data of peripheral white blood cells from beef heifers collected at the time of artificial insemination. Data Brief 18, 706-709 (2018).
42. Phillips, K. M. et al. Plasma metabolomic profiles differ at the time of artificial insemination based on pregnancy outcome, in Bos taurus beef heifers. Sci. Rep. 8(1), 13196 (2018).
43. Bormann, J. M., Totir, L. R., Kachman, S. D., Fernando, R. L. & Wilson, D. E. Pregnancy rate and first-service conception rate in Angus heifers. J. Anim. Sci. 84(8), 2022-2025 (2006).
44. Pryce, J. E., Royal, M. D., Garnsworthy, P. C. & Mao, I. L. Fertility in the high-producing dairy cow. Livest. Prod. Sci. 86(1-3), 125-135 (2004).
45. Wathes, D. C. et al. Influence of negative energy balance on cyclicity and fertility in the high producing dairy cow. Theriogenology 68(Suppl 1), S232-S241 (2007).
46. Diskin, M. G. & Kenny, D. A. Managing the reproductive performance of beef cows. Theriogenology 86(1), 379-387 (2016).
47. Randel, R. D. Nutrition and postpartum rebreeding in cattle. J. Anim. Sci. 68(3), 853-862 (1990).
48. Breuel, K. F. et al. Factors affecting fertility in the postpartum cow: Role of the oocyte and follicle in conception rate. Biol. Reprod. 48(3), 655-661 (1993).
49. Okano, A. & Tomizuka, T. Ultrasonic observation of postpartum uterine involution in the cow. Theriogenology 27(2), 369-376 (1987).
50. Sheldon, I. M., Williams, E. J., Miller, A. N., Nash, D. M. & Herath, S. Uterine diseases in cattle after parturition. Vet. J. 176(1), 115-121 (2008).
51. Sheldon, I. M., Molinari, P. C. C., Ormsby, T. J. R. & Bromfield, J. J. Preventing postpartum uterine disease in dairy cattle depends on avoiding, tolerating and resisting pathogenic bacteria. Theriogenology 150, 158-165 (2020).
52. Sheldon, I. M., Lewis, G. S., LeBlanc, S. & Gilbert, R. O. Defining postpartum uterine disease in cattle. Theriogenology 65(8), 1516-1530 (2006).
53. Crowley Jr., W. F., Pitteloud, N., & Seminara, S. New genes controlling human reproduction and how you find them. Trans. Am. Clin. Climatol. Assoc. 119, 29-37; discussion-8 (2008).
54. Yatsenko, S. A. & Rajkovic, A. Genetics of human female infertility. Biol. Reprod. 101(3), 549-566 (2019).
55. Mann, C. J. Observational research methods. Research design II: Cohort, cross sectional, and case-control studies. Emerg. Med. J. 20(1), 54-60 (2003).
56. Lima, F. S. et al. Hormonal manipulations in the 5-day timed artificial insemination protocol to optimize estrous cycle synchrony and fertility in dairy heifers. J. Dairy Sci. 96(11), 7054-7065 (2013).
57. Patterson, D., Kojima, F. & Smith, M. A review of methods to synchronize estrus in replacement beef heifers and postpartum cows. J. Anim. Sci. 81(14_suppl_2), E166-E177 (2003).
58. Wilson, C., Dias, N. W., Pancini, S., Mercadante, V. & Biase, F. H. Delayed processing of blood samples impairs the accuracy of mRNA-based biomarkers. Sci. Rep. 12(1), 8196 (2022).
59. Anderson, C. A. et al. Data quality control in genetic case-control association studies. Nat. Protoc. 5(9), 1564-1573 (2010).
60. Purcell, S. et al. PLINK: A tool set for whole-genome association and population-based linkage analyses. Am. J. Hum. Genet. 81(3), 559-575 (2007).
61. Hinrichs, A. S. et al. The UCSC genome browser database: Update 2006. Nucleic Acids Res. 34(90001), D590-D598 (2006).
62. Flicek, P. et al. Ensembl 2014. Nucleic Acids Res. 42(D1), D749-D755 (2014).
63. Pertea, M., Kim, D., Pertea, G. M., Leek, J. T. & Salzberg, S. L. Transcript-level expression analysis of RNA-seq experiments with HISAT, StringTie and Ballgown. Nat. Protoc. 11(9), 1650-1667 (2016).
64. Kim, D., Langmead, B. & Salzberg, S. L. HISAT: A fast spliced aligner with low memory requirements. Nat. Methods 12(4), 357-360 (2015).
65. Kim, D., Paggi, J. M., Park, C., Bennett, C. & Salzberg, S. L. Graph-based genome alignment and genotyping with HISAT2 and HISAT-genotype. Nat. Biotechnol. 37(8), 907-915 (2019).
66. Danecek, P. et al. Twelve years of SAMtools and BCFtools. Gigascience https://doi.org/10.1093/gigascience/giab008 (2021).
67. Li, H. et al. The sequence alignment/map format and SAMtools. Bioinformatics 25(16), 2078-2079 (2009).
68. Tischler, G. & Leonard, S. biobambam: Tools for read pair collation based algorithms on BAM files. Source Code Biol. Med. doi.org/10.1186/1751-0473-9-13 (2014).
69. Liao, Y., Smyth, G. K. & Shi, W. featureCounts: An efficient general purpose program for assigning sequence reads to genomic features. Bioinformatics 30(7), 923-930 (2014).
70. Orsburn, B. C. Proteome Discoverer—a community enhanced data processing suite for protein informatics. Proteomes https://doi.org/10.3390/proteomes9010015 (2021).
71. Che, R., Jack, J. R., Motsinger-Reif, A. A. & Brown, C. C. An adaptive permutation approach for genome-wide association study: Evaluation and recommendations for use. BioData Min. 7, 9 (2014).
72. Wellcome Trust Case Control C. Genome-wide association study of 14,000 cases of seven common diseases and 3000 shared controls. Nature. 447(7145), 661-678 (2007).
73. McCarthy, D. J. & Smyth, G. K. edgeR: A Bioconductor package for differential expression analysis of digital gene expression data. Bioinformatics 26, 139-140 (2010).
74. McCarthy, D. J., Chen, Y. & Smyth, G. K. Differential expression analysis of multifactor RNA-Seq experiments with respect to biological variation. Nucleic Acids Res. 40, 4288-4297 (2012).
75. Love, M. I., Huber, W. & Anders, S. Moderated estimation of fold change and dispersion for RNA-seq data with DESeq2. Genome Biol. 15, 550 (2014).
76. Storey, J. D. & Tibshirani, R. Statistical significance for genomewide studies. Proc. Natl. Acad. Sci. U.S.A. 100(16), 9440-9445 (2003).
77. Nakayasu, E. S. et al. Tutorial: Best practices and considerations for mass-spectrometry-based protein biomarker discovery and validation. Nat. Protoc. 16(8), 3737-3760 (2021).
78. Kalpić, D., Hlupić, N. & Lovrić, M. Student's t-tests. In International Encyclopedia of Statistical Science (ed. Lovric, M.) 1559-1563 (Springer, 2011).
79. Smyth, G. K. Limma: Linear Models for Microarray Data 397-420 (Springer, 2005).
80. Smyth, G. K. Linear models and empirical Bayes methods for assessing differential expression in microarray experiments. Stat. Appl. Genetics Mol. Biol. https://doi.org/10.2202/1544-6115.1027 (2004).
81. Phipson, B., Lee, S., Majewski, I. J., Alexander, W. S. & Smyth, G. K. Robust hyperparameter estimation protects against hyper-variable genes and improves power to detect differential expression. Ann. Appl. Stat. 10(2), 946-963 (2016).
82. Benjamini, Y. & Hochberg, Y. Controlling the false discovery rate-A practical and powerful approach to multiple testing. J. R. Stat. Soc. B Met. 57(1), 289-300 (1995).
83. Argelaguet, R. et al. Multi-omics factor analysis—A framework for unsupervised integration of multi-omics data sets. Mol. Syst. Biol. 14(6), e8124 (2018).
84. Argelaguet, R. et al. MOFA+: A statistical framework for comprehensive integration of multi-modal single-cell data. Genome Biol. 21(1), 111 (2020).
85. Orr, T. J. & Garland, T. Jr. Complex reproductive traits and whole-organism performance. Integr. Comp. Biol. 57(2), 407-422 (2017).
86. Hu, Z. L., Park, C. A. & Reecy, J. M. Bringing the Animal QTLdb and CorrDB into the future: Meeting new challenges and providing updated services. Nucleic Acids Res. 50(D1), D956-D961 (2022).
87. Yin, H., Hou, X., Zhang, T., Shi, L. & Su, Y. Q. Participation of EML6 in the regulation of oocyte meiotic progression in mice. J. Biomed. Res. 34(1), 44-53 (2019).
88. Yin, H. et al. Echinoderm microtubule associated protein like 1 is indispensable for oocyte spindle assembly and meiotic progression in mice. Front. Cell Dev. Biol. 9, 687522 (2021).
89. Walker, B. N. & Biase, F. H. The blueprint of RNA storages relative to oocyte developmental competence in cattle (Bos taurus). Biol Reprod. 102(4), 784-794 (2020).
90. de la Iglesia, R., Mansego, M. L., Sanchez-Muniz, F. J., Zulet, M. A. & Martinez, J. A. Arylesterase activity is associated with antioxidant intake and paraoxonase-1 (PON1) gene methylation in metabolic syndrome patients following an energy restricted diet. EXCLI J. 13, 416-426 (2014).
91. Pessentheiner, A. R. et al. APMAP interacts with lysyl oxidase-like proteins, and disruption of Apmap leads to beneficial visceral adipose tissue expansion. FASEB J. 31(9), 4088-4103 (2017).
92. Corton, M. et al. Proteomic analysis of human omental adipose tissue in the polycystic ovary syndrome using two-dimensional difference gel electrophoresis and mass spectrometry. Hum. Reprod. 23(3), 651-661 (2008).
93. Braschi, B. et al. Consensus nomenclature for dyneins and associated assembly factors. J. Cell Biol. https://doi.org/10.1083/jcb. 202109014 (2022).
94. Blyth, M. & Wellesley, D. Ectopic pregnancy in primary ciliary dyskinesia. J. Obstet Gynaecol. 28(3), 358 (2008).
95. Manenti, G. et al. Haplotype sharing suggests that a genomic segment containing six genes accounts for the pulmonary adenoma susceptibility 1 (Pas1) locus activity in mice. Oncogene 23(25), 4495-4504 (2004)
96. Kabbout, M. et al. ETS2 mediated tumor suppressive function and MET oncogene inhibition in human non-small cell lung cancer. Clin. Cancer Res. 19(13), 3383-3395 (2013).
97. Jia, G. et al. N6-methyladenosine in nuclear RNA is a major substrate of the obesity-associated FTO. Nat. Chem. Biol. 7(12), 885-887 (2011).
98. Mauer, J. et al. Reversible methylation of m(6)A(m) in the 5′ cap controls mRNA stability. Nature 541(7637), 371-375 (2017).
99. Zhao, X., Yang, Y., Sun, B. F., Zhao, Y. L. & Yang, Y. G. FTO and obesity: Mechanisms of association. Curr. Diab. Rep. 14(5), 486 (2014).
100. Willer, C. J. et al. Six new loci associated with body mass index highlight a neuronal influence on body weight regulation. Nat. Genet. 41(1), 25-34 (2009).
101. Thorleifsson, G. et al. Genome-wide association yields new sequence variants at seven loci that associate with measures of obesity. Nat. Genet. 41(1), 18-24 (2009).
102. Fischer, J. et al. Inactivation of the Fto gene protects from obesity. Nature 458(7240), 894-898 (2009).
103. Liu, A. L. et al. Association between fat mass and obesity associated (FTO) gene rs9939609 A/T polymorphism and polycystic ovary syndrome: A systematic review and meta-analysis. BMC Med. Genet. 18(1), 89 (2017).
104. Cheung, M. K., Gulati, P., O'Rahilly, S. & Yeo, G. S. FTO expression is regulated by availability of essential amino acids. Int. J. Obes. (Lond). 37(5), 744-747 (2013).
105. Boissel, S. et al. Loss-of-function mutation in the dioxygenase-encoding FTO gene causes severe growth retardation and multiple malformations. Am. J. Hum. Genet. 85(1), 106-111 (2009).
106. Michenet, A., Saintilan, R., Venot, E. & Phocas, F. Insights into the genetic variation of maternal behavior and suckling perfor-mance of continental beef cows. Genet Sel. Evol. 48(1), 45 (2016).
107. Albarran-Portillo, B. & Pollott, G. E. The relationship between fertility and lactation characteristics in Holstein cows on United Kingdom commercial dairy farms. J. Dairy Sci. 96(1), 635-646 (2013).
108. An, N., Yu, Z. & Yang, X. Expression differentiation is not helpful in identifying prognostic genes based on TCGA datasets. Mol. Ther. Nucleic Acids. 11, 292-299 (2018).
109. Lo, A., Chernoff, H., Zheng, T. & Lo, S. H. Why significant variables aren't automatically good predictors. Proc. Natl. Acad. Sci. U.S.A. 112(45), 13892-13897 (2015).

### Example 2

### Introduction

Heifers that calve at an optimum age have greater productivity and longevity in the herd (Lesmeister et al., 1973; Heinrichs, 1993; Hoffman, 1997; Cushman et al., 2013; Boulton et al., 2015; Damiran et al., 2018). Infertility or subfertility is a critical barrier to sustainable cattle production (Davis and White, 2020), including in heifers. For example, approximately 15% (Moorey and Biase, 2020) and 5% (Galliou et al., 2020) of beef and dairy heifers, respectively, do not calve at 24 mo of age. Therefore, identifying heifers with optimum fertility is a promising approach to improving sustainability in cattle production.

The broad detection and quantification of metabolites, namely metabolomics (Hollywood et al., 2006), can provide critical biological insights into multiple aspects of the physiology in complex organisms (Liu and Locasale, 2017). Multiple studies have used metabolomics approaches to understand metabolic profiles associated with nutritional conditions (Zhang et al., 2013, 2019; Li et al., 2014; Leal et al., 2021; Horn et al., 2022; Jorge-Smeding et al., 2022) and female reproductive phenotypes (Bender et al., 2010; Phillips et al., 2018; Read et al., 2021) in cattle. The plasma component of the blood is a major carrier of metabolites produced in mammals, and 3,126 of those metabolites have been quantified in the plasma of human blood (serummetabolome.ca/statistics, 29 Dec. 2023, (Psychogios et al., 2011)). Because blood carries metabolites used, secreted or disposed by nearly all organs, this connective tissue is valuable for the discovery of molecular signatures, including metabolites, linked to complex phenotypes in a biological system (Qiu et al., 2023).

It was reported that transcript abundances of specific genes are altered in the circulating white blood cells of crossbred heifers of different fertility potential (Dickinson and Biase, 2018; Dickinson et al., 2018; Moorey et al., 2020), and recently reported genotypes, transcript and protein abundances in the bloodstream associated with fertility in Bos taurus purebred heifers (Marrella and Biase, 2023). Here, Applicant tested the hypothesis that specific metabolites would be differentially abundant in plasma of Holstein and Angus heifers (Bos taurus) with different fertility potentials. The objective of this study was to determine the metabolite profiles of heifers at the time of artificial insemination and contrast metabolite abundances between heifers classified as fertile or sub-fertile.

### Materials and Methods

### Heifer Classification

Applicant collected blood samples from purebred Angus heifers (n=12), averaging 14 mo in age, at their first artificial insemination service. Heifers were subjected to a 7-Day Co-Synch+CIDR estrus synchronization protocol prior to breeding. Briefly, heifers were administered an intramuscular (IM) injection of gonadotropin-releasing hormone (GnRH, 100 g; Factrel; Zoetis Inc.) on day 0, followed by the insertion of a controlled internal drug release (CIDR, 1.38 g Progesterone; Eazi-Breed CIDR; Zoetis Inc.). On day 7, the CIDR was removed and an injection of prostaglandin F2 alpha (PGF2a, 25 g; Lutalyse; Zoetis Inc.) was delivered. Fixed-time artificial insemination was performed 54±2 h following CIDR removal along a second injection of GnRH.

Additionally, Applicant collected blood samples from purebred Holstein (n=10) heifers, averaging 12 mo in age, at the time of the first artificial insemination service. Heifers were enrolled in a 5-d CIDR-Synch protocol before artificial insemination. Briefly, an IM injection of GnRH was delivered on Day 0 with the insertion of a CIDR device. The CIDR device was removed on Day 5, followed by an IM injection of PGF2a. A second injection of PGF2α_was administered 24 hours later. Then, timed AI was performed with a second GnRH injection on Day 8.

Heifers were identified as Fertile (Holstein, n=5; Angus, n=5) or Sub-Fertile (Holstein, n=5; Angus, n=7) based on their pregnancy outcome, following similar criteria used previously (Dickinson et al., 2018; Moorey et al., 2020). Fertile animals were identified as those who became pregnant and subsequently delivered a calf following the first insemination service. Angus heifers were categorized as Sub-Fertile after failing to achieve pregnancy following two insemination services and exposure to a bull for natural breeding. Holstein heifers were identified as Sub-Fertile after needing four or more artificial inseminations.

Heifers were synchronized with protocols that have been identified by prior research to have high success for a heifer to become pregnant to artificial insemination (Patterson et al., 2003; Lima et al., 2013). Hence the different protocols for beef and dairy heifers. The criteria for classification were different for each group due to differences in management that are inherent to beef and dairy replacement heifers. Most importantly, each heifer had multiple opportunities to become pregnant before being classified as sub-fertile. The heifers utilized in this study were not part of a nutritional experiment, and thus nutrition was not accounted as a variable nor was it a factor in the selection of heifers. All dairy heifers were raised with equivalent exposure to feed. Similarly, all beef heifers were raised with equivalent exposure to feed.

### Blood Sample Collection and Plasma Separation

Ten millimeters of blood were drawn from each animal by venipuncture of the jugular vein using 18 mg K2 EDTA vacutainers (Becton, Dickinson, and Company). The tubes were inverted for proper mixing with the anticoagulant and then immediately placed on ice until further processing.

Applicant processed the blood samples following procedures described elsewhere (Dickinson et al., 2018; Moorey et al., 2020; Wilson et al., 2022) within 3 h of sampling (Wilson et al., 2022). Tubes containing whole blood samples were centrifuged for 25 min at 4° C. and 2,000×g. Two milliliters of plasma were aspirated and centrifuged at 1,000×g for 10 min at 4° C. to pellet any remaining cells. The supernatant was deposited in a cryotube and snap-frozen with liquid nitrogen. Samples were then stored at −80° C. until further processing.

### Metabolome Data Collection

Ultra-high-performance liquid chromatography-high resolution mass spectrometry (UHPLC-HRMS) analysis was performed on plasma samples at the Biological and Small Molecule Mass Spectrometry Core (RRID: SCR_021368) at the University of Tennessee, Knoxville, as previously described (Horn et al., 2022). One hundred microliter aliquots of plasma samples were thawed at 4° C. for 30 min, and metabolites within each sample were extracted using a 40:40:20 methanol/acetonitrile/water solution with 0.1 M formic acid (Rabinowitz and Kimball, 2007; Lu et al., 2010).

Extraction solvent (1.3 mL) was added to the samples which underwent agitation and vortexing before samples were then chilled at −20° C. for 20 min. Once samples were properly chilled, the tubes were centrifuged at 4° C. and 15,000 rpm for 5 min to form pellets and remove debris from the sample. The supernatant was transferred to a 2-mL microcentrifuge tube and stored at −20° C. The pellet was resuspended in 0.2 mL of extraction solvent and the tubes were then re-submitted to agitation and vortexing, chilling, and centrifugation as described above. The supernatant obtained in the second round of extraction was added to supernatant collected during the first round of extraction.

The first tube set with the final pellet was then discarded. The second set of tubes containing the supernatant underwent drying using nitrogen gas. Once dried, tubes were filled with 300 μL of LCMS grade water for sample resuspension. The resuspended samples were vortexed and centrifuged at 4° C. and 15,000 rpm for 5 min before transferring an aliquot to new autosampler vials for UHPLC-HRMS analysis. Metabolites present in samples were separated using chromatography column (Synergi Hydro RP, 2.5 μm, 100 mm×2.0 mm column; Phenomenex, Torrance, CA, United States) which was maintained at 25° C. The mobile phase solvents used to elute metabolites were 1) 97:3 LCMS grade water:methanol with 15 mM acetic acid and 11 mM tributylamine and 2) 100% LCMS grade methanol. At a flow rate of 0.2 mL/min, the solvent gradient was 1) 100% and 2) 0% from 0 to 5 min, 1) 80% and 2) 20% from 5 to 13 min, 1) 45% and 2) 55% from 13 to 15.5 min, 1) 5% and 2) 95% from 15.5 to 19 min, and 1) 100% and 2) 0% from 19 to 25 min. An Exactive Plus Orbitrap mass spectrometer (Thermo Fisher Scientific, Waltham, MA, USA) with an electrospray ionization probe attached was used for full scan mass analysis, operating in negative polarity mode with a scan range between 72 and 1,000 m/z, a resolution of 140,000, and an acquisition gain control of 3×10⁶.

The tandem liquid chromatography and mass spectrometry analysis generated Xcalibur (RAW) files which were converted to an open source mzML format (msconvert software; ProteoWizard package) to enable data centroiding. The centroided data were processed using the Metabolomic Analysis and Visualization Engine (MAVEN; mzroll software, Princeton University) to identify metabolites that are present in the samples by comparing their retention time and mass-to-charge ratio with an in-house standard library. Peak identification for each metabolite is carried out using a variety of factors including peak shape, retention time, mass-to-charge ratio, signal-to-noise-ratio and this is validated using the natural abundance of isotopes in the compound The program then generates pre-processed peak data tables. Normalized data were obtained by dividing the raw peak areas by the volume of sample used for metabolite extraction.

### Analysis of Metabolome Data

First, Applicant transformed the normalized metabolome data using logarithm (Log 2(x)). In order to increase the robustness of these findings we analyzed the metabolome data using two different univariate methodologies (Bartel et al., 2013). Applicant analyzed the transformed data using a generalized linear model (Nakayasu et al., 2021) using the “lm” function from the “stats” R package, which included the fertility group (Fertile or Sub-Fertile), breed (Angus or Holstein) as fixed effects. Then, Applicant used the function “emmeans”, to test the significance of the difference of adjusted means (μ) (H0:μ1=μ2, H1:μ1≠μ2) with the Student's t test (Kalpić et al., 2011), to calculate the estimated differences in metabolite abundance. Applicant also analyzed the data using the R package ‘limma’ (Smyth, 2005; Ritchie et al., 2015). We accounted for the same independent variables mentioned above (fertility group and breed) to fit the model and then tested for a differential abundance of the metabolites using the empirical Bayes Statistics implemented in the function “eBayes” (Smyth, 2004; Phipson et al., 2016). Although it was not the main focus of the work, we also carried out a t-test (Welch, 1947) of the metabolites within each breed to compare samples in both categories of fertility (Fertile or Sub-Fertile), using the function “t_test” and “cohens_d” in the “rstatix” R package. In all analyses, we adjusted the nominal P values using false discovery rate (FDR) (Benjamini and Hochberg, 1995). Significance was inferred if FDR<0.1.

### Results and Discussion

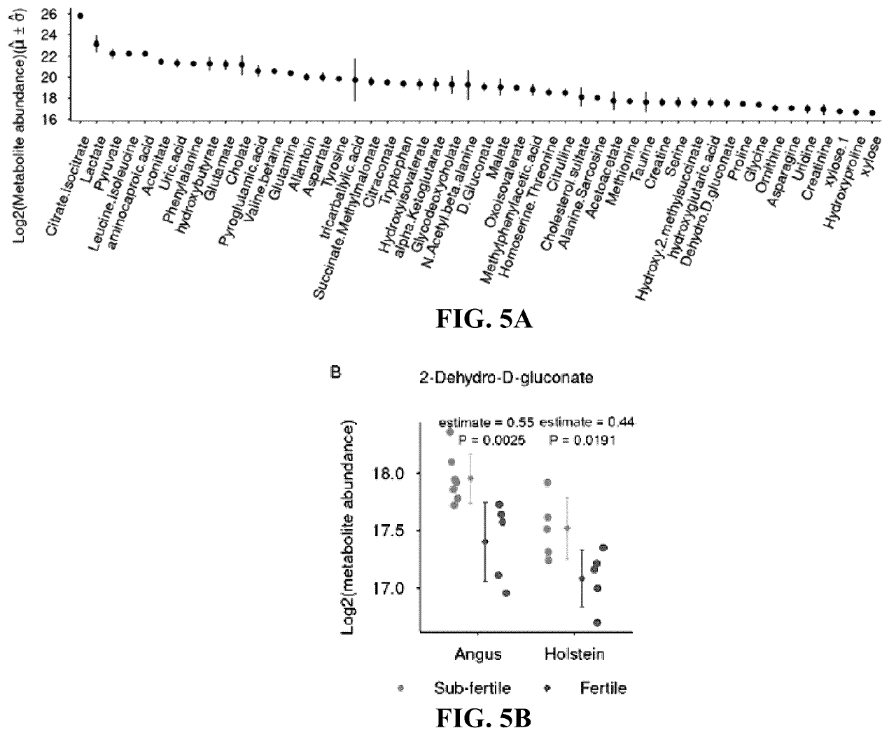

Applicant collected data for 140 metabolites present in the plasma of heifers (Supplemental Table S1 of Marrella et al., “Higher abundance of 2-dehydro-D-gluconate in the plasma of sub-fertile or infertile Bos taurus heifers” J. Anim. Sci. 102, skae126 (2024), which is incorporated by reference herein). The majority of metabolites identified and quantified were “amino acids; amino acid precursors and derivatives” (n=40), followed by “nucleosides, nucleotides and analogues” (n=21). In addition, there were compounds classified as “amino acid metabolism” (n=12), “carbohydrates and carbohydrate conjugates” (n=11) and “lipids and lipid-like molecules” (n=11), among other functional classification. Interestingly, the top three most abundant compounds participate in energy related metabolic pathways (Citrate/isocitrate, TCA cycle; Lactate and Pyruvate, Glycolysis & Gluconeogenesis) (FIG. 5A).

The contrast of metabolites abundance between heifers of different genetic background resulted in the identification of 2-dehydro-d-gluconate (C₆H₁₀O₇) as differentially abundant between the two groups of fertility (fold change=1.41, P=0.0003, FDR<0.1; Supplemental Table S2 of Marrella et al., “Higher abundance of 2-dehydro-D-gluconate in the plasma of sub-fertile or infertile Bos taurus heifers” J. Anim. Sci. 102, skae126 (2024), which is incorporated by reference herein). The analysis within breeds also confirmed that the metabolite 2-dehydro-d-gluconate was more abundant in the plasma of sub-fertile Angus and Holstein heifers at a similar fold change (FIG. 5B).

In a similar work (Phillips et al., 2018), 15 metabolites were identified as significantly associated with fertility in heifers. Nine of those metabolites were also identified in our samples (asparagine, cystine, glutamine, histidine, kynurenine, lysine, methionine, ornithine, and tryptophan), but were not statistically different in our tests. One possible source of difference in the results for the nine metabolites known to overlap with our dataset is that Phillips et al. sampled Angus crossed heifers, whereas we worked with purebred Angus and Holstein heifers. The analysis of Angus heifers alone resulted in no statistically significant differences observed for all metabolites in our dataset (Supplementary Table S2 of Marrella et al., “Higher abundance of 2-dehydro-D-gluconate in the plasma of sub-fertile or infertile Bos taurus heifers” J. Anim. Sci. 102, skae126 (2024), which is incorporated by reference herein). This discrepancy in results between two studies opens the possibility for an environmental interaction in metabolite abundance and fertility in heifers. Considering that all nine metabolites mentioned above are amino acids, we may reason that regional differences in climate and forage availability or supplementation to meet nutrient requirements of the heifers between the deep south and Appalachian regions of Alabama and Virginia, could have contributed to different outcomes for these nine metabolites. Such environmental interactions could have undoubtedly influenced metabolite levels and their discriminatory capacity between heifers of high- and low-fertility potential.

It is unclear whether 2-dehydro-d-gluconate was identified in the study by Phillips et al. for a comparison with our results. Regardless, the abundance of the metabolite 2-dehydro-d-gluconate has been connected with a fertility related phenotype. In beef cows, the bigger the preovulatory follicle, at the time of administration of GnRH to induce ovulation, the greater the abundance of 2-dehydro-d-gluconate (Read et al., 2021). This finding is relevant because there is a greater chance of a pregnancy to be established when ovulation occurs if the dominant follicle is 14.7 mm in diameter relative to if the dominant follicles <12.1 mm in diameter at the time of induction of ovulation (Perry et al., 2005). Also notable, in the study by (Read et al., 2021), d-gluconate was positively correlated with follicle diameter at the time of administration of GnRH to induce ovulation. d-Gluconate had the second smallest P-values in our analysis (P=0.0061), but did not reach the FDR for statistical inference of significant difference between fertility groups. Our results allied with the literature show an important connection of 2-dehydro-d-gluconate with fertility in Bos taurus heifers.

The link between greater quantities of 2-dehydro-d-gluconate, a compound in the pentose phosphate pathway, in the blood of sub-fertile heifers is not trivial. One possible explanation is that fertile heifers could have a greater abundance of circulating estradiol (Northrop et al., 2018) which would stimulate more activity in pentose phosphate pathway (Zheng et al., 2021) in fertile heifers relative to sub-fertile heifers. Consequently, the greater activity of pentose phosphate pathway would cause more utilization of 2-dehydro-d-gluconate, and thus the measured lower abundance 2-dehydro-d-gluconate in the bloodstream. Another plausible explanation involves the fact that microorganisms in the gut microbiome produce and utilize 2-Dehydro-D-gluconate, and thus variations in the microbiome could also be a source differential abundance (Diener et al., 2022) of 2-Dehydro-D-gluconate in the plasma of heifers of different fertility potential. Both possibilities were not tested in the current study but may warrant further investigation.

In summary, we report the profile of 140 metabolites in plasma sampled from Angus and Holstein heifers (Bos taurus). A robust statistical analysis identified a greater abundance of the metabolite 2-Dehydro-D-gluconate in heifers that failed to produce a calf after multiple breeding attempts. Considering the highly variable nature of metabolites in the bloodstream (Qiu et al., 2023), the differential abundance of 2-Dehydro-D-gluconate in two distinct groups of heifers enhance the robustness of this finding. Our study is the second to show an association of 2-Dehydro-D-gluconate with fertility phenotype in Bos taurus which further strengthen our findings. Thus, further research may deepen our understanding of the connection of this metabolite and possibly the pentose phosphate pathway with female fertility.

### REFERENCES FOR EXAMPLE 2

Bartel, J., J. Krumsiek, and F. J. Theis. 2013. Statistical methods for the analysis of high-throughput metabolomics data. Comput. Struct. Biotechnol. J. 4:e201301009. doi:10.5936/csbj.201301009
Bender, K., S. Walsh, A. C. Evans, T. Fair, and L. Brennan. 2010. Metabolite concentrations in follicular fluid may explain differences in fertility between heifers and lactating cows. Reproduction 139:1047-1055. doi:10.1530/REP-10-0068
Benjamini, Y., and Y. Hochberg. 1995. Controlling the false discovery rate—a practical and powerful approach to multiple testing. J. R. Stat. Soc. Ser. B Methodol. 57:289-300. doi: 10.1111/j.2517-6161.1995.tb02031.x
Boulton, A. C., J. Rushton, and D. C. Wathes. 2015. A study of dairy heifer rearing practices from birth to weaning and their associated costs on UK dairy farms. Open J. Anim. Sci. 05:185-197. doi:10.4236/ojas.2015.52021
Cushman, R. A., L. K. Kill, R. N. Funston, E. M. Mousel, and G. A. Perry. 2013. Heifer calving date positively influences calf weaning weights through six parturitions. J. Anim. Sci. 91:4486-4491. doi:10.2527/jas.2013-6465
Damiran, D., K. A. Larson, L. T. Pearce, N. E. Erickson, and B. H. A. Lardner. 2018. Effect of calving period on beef cow longevity and lifetime productivity in western Canada. Transl. Anim. Sci. 2:S61-S65. doi:10.1093/tas/txy020
Davis, T. C., and R. R. White. 2020. Breeding animals to feed people: the many roles of animal reproduction in ensuring global food security. Theriogenology 150:27-33. doi:10.1016/j.theriogenology.2020.01.041
Dickinson, S. E., and F. H. Biase. 2018. Transcriptome data of peripheral white blood cells from beef heifers collected at the time of artificial insemination. Data Brief 18:706-709. doi:10.1016/j.dib.2018.03.062
Dickinson, S. E., B. A. Griffin, M. F. Elmore, L. Kriese-Anderson, J. B. Elmore, P. W. Dyce, S. P. Rodning, and F. H. Biase. 2018. Transcriptome profiles in peripheral white blood cells at the time of artificial insemination discriminate beef heifers with different fertility potential. BMC Genomics 19:129. doi:10.1186/s12864-018-4505-4
Diener, C., C. L. Dai, T. Wilmanski, P. Baloni, B. Smith, N. Rappaport, L. Hood, A. T. Magis, and S. M. Gibbons. 2022. Genome-microbiome interplay provides insight into the determinants of the human blood metabolome. Nat. Metab. 4:1560-1572. doi:10.1038/s42255-022-00670-1
Galliou, J. M., J. N. Kiser, K. F. Oliver, C. M. Seabury, J. G. N. Moraes, G. W. Burns, T. E. Spencer, J. Dalton, and H. L. Neibergs. 2020. Identification of loci and pathways associated with heifer conception rate in U.S. Holsteins. Genes (Basel) 11:767. doi:10.3390/genes11070767
Heinrichs, A. J. 1993. Raising dairy replacements to meet the needs of the 21st century. J. Dairy Sci. 76:3179-3187. doi:10.3168/jds.S0022-0302(93)77656-0
Hoffman, P. C. 1997. Optimum body size of Holstein replacement heifers. J. Anim. Sci. 75:836-845. doi:10.2527/1997.753836x
Hollywood, K., D. R. Brison, and R. Goodacre. 2006. Metabolomics: current technologies and future trends. Proteomics 6:4716-4723. doi:10.1002/pmic.200600106
Horn, E. J., C. C. Read, J. L. Edwards, F. N. Schrick, J. D. Rhinehart, R. R. Payton, S. R. Campagna, J. L. Klabnik, H. M. Clark, P. R. Myer, et al. 2022. Preovulatory follicular fluid and serum metabolome profiles in lactating beef cows with thin, moderate, and obese body condition. J. Anim. Sci. 100:skac152. doi:10.1093/jas/skac152
Jorge-Smeding, E., S. Polakof, M. Bonnet, S. Durand, D. Centeno, M. Petera, S. Taussat, and G. Cantalapiedra-Hijar. 2022. Untargeted metabolomics confirms the association between plasma branched chain amino acids and residual feed intake in beef heifers. PLoS One 17:e0277458. doi:10.1371/journal.pone.0277458
Kalpić, D., N. Hlupid, and M. Lovrid. 2011. Student's t-tests. In: Lovric, M., editor. International encyclopedia of statistical science. Berlin, Heidelberg: Springer Berlin Heidelberg; p. 1559-1563.
Leal, L. N., J. Doelman, B. R. Keppler, M. A. Steele, and J. Martin-Tereso. 2021. Preweaning nutrient supply alters serum metabolomics profiles related to protein and energy metabolism and hepatic function in Holstein heifer calves. J. Dairy Sci. 104:7711-7724. doi:10.3168/jds.2020-19867
Lesmeister, J. L., P. J. Burfening, and R. L. Blackwell. 1973. Date of first calving in beef cows and subsequent calf production. J. Anim. Sci. 36:1-6. doi:10.2527/jas1973.3611
Li, Y., C. Xu, C. Xia, H. Zhang, L. Sun, and Y. Gao. 2014. Plasma metabolic profiling of dairy cows affected with clinical ketosis using LC/MS technology. Vet. Q. 34:152-158. doi:10.1080/01652176.2014.962116
Lima, F. S., E. S. Ribeiro, R. S. Bisinotto, L. F. Greco, N. Martinez, M. Amstalden, W. W. Thatcher, and J. E. P. Santos. 2013. Hormonal manipulations in the 5-day timed artificial insemination protocol to optimize estrous cycle synchrony and fertility in dairy heifers. J. Dairy Sci. 96:7054-7065. doi:10.3168/jds.2013-7093
Liu, X., and J. W. Locasale. 2017. Metabolomics: a primer. Trends Biochem. Sci. 42:274-284. doi:10.1016/j.tibs.2017.01.004
Lu, W., M. F. Clasquin, E. Melamud, D. Amador-Noguez, A. A. Caudy, and J. D. Rabinowitz. 2010. Metabolomic analysis via reversed-phase ion-pairing liquid chromatography coupled to a stand alone orbitrap mass spectrometer. Anal. Chem. 82:3212-3221. doi:10.1021/ac902837x
Marrella, M. A., and F. H. Biase. 2023. A multi-omics analysis identifies molecular features associated with fertility in heifers (Bos taurus). Sci. Rep. 13:12664. doi:10.1038/s41598-023-39858-0
Moorey, S. E., and F. H. Biase. 2020. Beef heifer fertility: importance of management practices and technological advancements. J. Anim. Sci. Biotechnol. 11:97. doi:10.1186/s40104-020-00503-9
Moorey, S. E., B. N. Walker, M. F. Elmore, J. B. Elmore, S. P. Rodning, and F. H. Biase. 2020. Rewiring of gene expression in circulating white blood cells is associated with pregnancy outcome in heifers (Bos taurus). Sci. Rep. 10:16786. doi:10.1038/s41598-020-73694-w
Nakayasu, E. S., M. Gritsenko, P. D. Piehowski, Y. Gao, D. J. Orton, A. A. Schepmoes, T. L. Fillmore, B. I. Frohnert, M. Rewers, J. P. Krischer, et al. 2021. Tutorial: best practices and considerations for mass-spectrometry-based protein biomarker discovery and validation. Nat. Protoc. 16:3737-3760. doi:10.1038/s41596-021-00566-6
Northrop, E. J., J. J. J. Rich, R. A. Cushman, A. K. McNeel, E. M. Soares, K. Brooks, T. E. Spencer, and G. A. Perry. 2018. Effects of preovulatory estradiol on uterine environment and conceptus survival from fertilization to maternal recognition of pregnancy. Biol. Reprod. 99:629-638. doi:10.1093/biolre/ioy086
Patterson, D., F. Kojima, and M. Smith. 2003. A review of methods to synchronize estrus in replacement beef heifers and postpartum cows. J. Anim. Sci. 81:E166-E177. doi:10.2527/2003.8114_suppl_2E166x
Perry, G. A., M. F. Smith, M. C. Lucy, J. A. Green, T. E. Parks, M. D. MacNeil, A. J. Roberts, and T. W. Geary. 2005. Relationship between follicle size at insemination and pregnancy success. Proc. Natl. Acad. Sci. U.S.A. 102:5268-5273. doi:10.1073/pnas.0501700102
Phillips, K. M., C. C. Read, L. A. Kriese-Anderson, S. P. Rodning, T. D. Brandebourg, F. H. Biase, M. L. Marks, J. B. Elmore, M. K. Stanford, and P. W. Dyce. 2018. Plasma metabolomic profiles differ at the time of artificial insemination based on pregnancy outcome, in Bos taurus beef heifers. Sci. Rep. 8:13196. doi:10.1038/s41598-018-31605-0
Phipson, B., S. Lee, I. J. Majewski, W. S. Alexander, and G. K. Smyth. 2016. Robust hyperparameter estimation protects against hypervariable genes and improves power to detect differential expression. Ann. Appl. Stat. 10:946-963. doi:10.1214/16-AOAS920
Psychogios, N., D. D. Hau, J. Peng, A. C. Guo, R. Mandal, S. Bouatra, I. Sinelnikov, R. Krishnamurthy, R. Eisner, B. Gautam, et al. 2011. The human serum metabolome. PLoS One 6:e16957. doi:10.1371/journal.pone.0016957
Qiu, S., Y. Cai, H. Yao, C. Lin, Y. Xie, S. Tang, and A. Zhang. 2023. Small molecule metabolites: discovery of biomarkers and therapeutic targets. Signal Transduct Target Ther. 8:132. doi:10.1038/s41392-023-01399-3
Rabinowitz, J. D., and E. Kimball. 2007. Acidic acetonitrile for cellular metabolome extraction from Escherichia coli. Anal. Chem. 79:6167-6173. doi:10.1021/ac070470c
Read, C. C., L. Edwards, N. Schrick, J. D. Rhinehart, R. R. Payton, S. R. Campagna, H. F. Castro, J. L. Klabnik, E. J. Horn, and S. E. Moorey. 2021. Correlation between pre-ovulatory follicle diameter and follicular fluid metabolome profiles in lactating beef cows. Metabolites 11:623. doi:10.3390/metabo11090623
Ritchie, M. E., B. Phipson, D. Wu, Y. Hu, C. W. Law, W. Shi, and G. K. Smyth. 2015. limma powers differential expression analyses for RNA-sequencing and microarray studies. Nucleic Acids Res. 43:e47. doi:10.1093/nar/gkv007
Smyth, G. K. 2004. Linear models and empirical bayes methods for assessing differential expression in microarray experiments. Stat. Appl. Genet. Mol. Biol. 3:Article3. doi:10.2202/1544-6115.1027
Smyth, G. K. 2005. Limma: linear models for microarray data, Bioinformatics and computational biology solutions using R and Bioconductor. New York, NY: Springer; p. 397-420.
Welch, B. L. 1947. The generalisation of student's problems when several different population variances are involved. Biometrika 34:28-35. doi:10.1093/biomet/34.1-2.28
Wilson, C., N. W. Dias, S. Pancini, V. Mercadante, and F. H. Biase. 2022. Delayed processing of blood samples impairs the accuracy of mRNA-based biomarkers. Sci. Rep. 12:8196. doi:10.1038/s41598-022-12178-5
Zhang, H., L. Wu, C. Xu, C. Xia, L. Sun, and S. Shu. 2013. Plasma metabolomic profiling of dairy cows affected with ketosis using gas chromatography/mass spectrometry. BMC Vet. Res. 9:186. doi:10.1186/1746-6148-9-186
Zhang, J., H. Shi, S. Li, Z. Cao, H. Yang, and Y. Wang. 2019. Integrative hepatic metabolomics and proteomics reveal insights into the mechanism of different feed efficiency with high or low dietary forage levels in Holstein heifers. J. Proteomics 194:1-13. doi:10.1016/j.jprot.2018.12.026
Zheng, Y., Y. Zhu, T. Zhuge, B. Li, and C. Gu. 2021. Metabolomics analysis discovers estrogen altering cell proliferation via the pentose phosphate pathway in infertility patient endometria. Front Endocrinol. (Lausanne) 12:791174. doi:10.3389/fendo.2021.791174

Further embodiments are illustrated in the following Examples which are given for illustrative purposes only and are not intended to limit the scope of the invention.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and can be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. A method of assessing fertility fitness in a female mammal, the method comprising:
in one or more samples collected from the female mammal
(a) detecting one or more single nucleotide polymorphisms (SNPs) set forth in
(i) Supplementary Information 3;
(ii) BovineHD500034888, BovineHD500034891, BovineHD500034893, BovineHD500034894, BovineHD500034895, BovineHD500034896, BovineHD500034897, BovineHD500034898, BovineHD500034899, BovineHD1200026258, BovineHD1200026258, BovineHD2700000503, BovineHD2500009748, BovineHD2600007565, BovineHD2600007566, BovineHD1600016722, BovineHD1300006153, BovineHD2300004608, BovineHD0500034892, BovineHD0300007001, BovineHD1200026258, BovineHD2700000503, BovineHD1600016722, BovineHD0500034794, BovineHD0100008466, BovineHD2300004608, BovineHD2100009580, BovineHD2100009629, BovineHD2600011917, BovineHD0500035192, BovineHD0500035196; and/or
(iii) BovineHD500034888, BovineHD500034891, BovineHD500034893, BovineHD500034894, BovineHD500034895, BovineHD500034896, BovineHD500034897, BovineHD500034898, BovineHD500034899, BovineHD1200026258;
(b) determining an amount of a transcript of one or more genes set forth in
(iv) Supplementary Information 4;
(v) ENSBTAG00000021346, ENSBTAG00000047139, ENSBTAG0000003279, ENSBTAG0000015041, ENSBTAG0000007389, ENSBTAG0000000810, ENSBTAG0000040199, ENSBTAG0000002972, ENSBTAG0000004278, ENSBTAG0000019300, ENSBTAG00000012263, ENSBTAG00000018339, ENSBTAG00000007566, ENSBTAG00000015061, ENSBTAG00000054926, ENSBTAG00000018655, ENSBTAG00000021346, ENSBTAG00000040065, ENSBTAG00000009479, ENSBTAG0000005211, ENSBTAG00000018339, ENSBTAG00000054926, ENSBTAG00000040065, ENSBTAG00000009479, ENSBTAG00000047139, ENSBTAG00000003279, ENSBTAG00000015041, ENSBTAG00000007398, ENSBTAG00000000810, ENSBTAG00000040199;
(vi) ENSBTAG00000012263, ENSBTAG00000018339, ENSBTAG00000007566, ENSBTAG00000015061, ENSBTAG00000054926, ENSBTAG00000018655, ENSBTAG00000021346, ENSBTAG00000040065, ENSBTAG00000009479, ENSBTAG0000005211; and/or from
(vii) one or more genes selected from the group consisting of ArfGAP with SH3 domain, ankyrin repeat and PH domain 3 (ASAP3), ATP synthase membrane subunit c locus 1 (ATP5MC1), Centrosomal protein 170 (CEP170), Myeloid derived growth factor (MYDGF), Coiled-coil domain containing 34 (CCDC34), RAD51 associated protein 1 (RAD51AP1), and Ubiquinol-cytochrome c reductase complex III subunit VII (UQCRQ);
(c) determining an amount of one or more proteins set forth in
(viii) Table 2;
(ix) P00744; P02768, Q2HJF0, Q95121, A5D798, F1MZ96, Q2KIX7, G1K122, F6QND5, Q5E9E3, P19034, P02768, F1N102, F1MZ96, G3X6N3, Q2KIX7, G5E5V0, G3MY71, G1K122, Q5EA67, P19034, F1MYX5, P00744, AOA6B9SCM2, P02768, Q2HJF0, F1N102, Q95121, P80109, A5D798;
(x) P19034, F1MYX5, P00744, AOA6B9SCM2, P02768, Q2HJF0, F1N102, Q95121, P80109, A5D798; and/or
(xi) from one or more proteins selected from Apolipoprotein C-II (APOC2), Lymphocyte cytosolic protein 1 (LCP1), Vitamin K-dependent protein Z (PROZ), Albumin (ALB), Serotransferrin-like (LOC525947), Complement component C8 beta chain (C8B), Pigment epithelium-derived factor (SERPINF1), Phosphatidylinositol-glycan-specific phospholipase D (GPLD1), Alpha-ketoglutarate-dependent dioxygenase FTO (FTO); or
(e) from one or more metabolites, optionally wherein the metabolite is 2-Dehydro-D-gluconate; or
(d) any combination of (a)-(d).

2. A method of assessing fertility fitness in a female mammal, the method comprising:
in one or more samples collected from the female mammal
(a) detecting the presence one or more single nucleotide polymorphisms (SNPs) selected from rs110918927, chr12: 85648422 and rs109366560, chr11:37666527;
(b) detecting and/or quantifying an amount of a transcript of adipocyte plasma membrane associated protein (APMAP), dynein axonemal intermediate chain 7 (DNAI7), or both;
(c) detecting and/or quantifying an amount of Alpha-ketoglutarate-dependent dioxygenase FTO (FTO);
(d) detecting and/or quantifying an amount of 2-Dehydro-D-gluconate; or
(e) any combination of (a)-(e).

3. The method of any one of aspects 1-2, whereby detecting and/or quantifying (a), (b), (c), (d) or any combination thereof determines that the female mammal is fertile or subfertile.

4. The method of any one of aspects 1-3, wherein the female mammal is fertile when the female mammal is (a) homozygous for allele A at rs110918927, chr12: 85648422, (b) is homozygous for allele G at rs109366560, chr11:37666527 (c) has a greater amount of transcript from APMAP as compared to a suitable control, (d) has a lesser amount of transcript of DNAI7 than a suitable control, (e) has a greater amount of FTO, (f) has a greater amount of 2-Dehydro-D-gluconate than a suitable control; or (g) any combination of (a)-(f).

5. The method of any one of aspects 1-4, wherein the female mammal is subfertile when the female mammal (a) has at least one G allele at rs110918927, chr12: 85648422, (b) is homozygous for allele G at rs109366560, chr11:37666527, (c) has a lesser amount of transcript from APMAP as compared to a suitable control, (d) has a greater amount of transcript of DNAI7 than a suitable control, (e) has a lesser amount of FTO as compared to a suitable control, (f) has a greater amount of 2-Dehydro-D-gluconate than a suitable control; or (g) any combination of (a)-(f).

6. The method of any one of aspects 1-5, wherein the female mammal is subfertile when the female mammal (a) has one A allele and one G allele or is homozygous for the G allele at rs110918927, chr12: 85648422, (b) is homozygous for allele G at rs109366560, chr11:37666527, (c) has a lesser amount of transcript from APMAP as compared to a suitable control, (d) has a greater amount of transcript of DNAI7 than a suitable control, (e) has a lesser amount of FTO as compared to a suitable control, (f) has a greater amount of 2-Dehydro-D-gluconate than a suitable control; or (g) any combination of (a)-(f).

7. The method of any one of aspects 1-6, wherein the femail mammal is subfertile when the female mammal has more 2-Dehydro-D-gluconate than a suitable control, optionally wherein the suitable control is a fertile female mammal.

8. The method of any one of aspects 1-7, wherein the female mammal is a non-human animal.

9. The method of any one of aspects 1-8, wherein the female mammal is a bovine, equine, ovine, porcine, canine, or feline.

10. The method of any one of aspects 1-9, wherein the female mammal is a human.

11. The method of any one of aspects 1-10, wherein the female mammal is pre-pubertal, is pubertal, or is sexually mature.

12. The method of any one of aspects 1-11, wherein the one or more samples comprise a bodily fluid.

13. The method of any one of aspects 1-12, wherein the one or more samples comprises blood or component thereof.

14. The method of any one of aspects 1-13, wherein the one or more samples comprises plasma, buffy coat, or both.

15. The method of any one of aspects 1-14, wherein the one or more samples comprises cells.

16. The method of aspect 15, wherein the cells are white blood cells.

17. The method of any one of aspects 1-16, wherein the one or more samples used for detecting one or more single nucleotide polymorphisms (SNPs) comprises cells.

18. The method of any one of aspects 1-17, wherein the one or more samples used for detecting and/or quantifying an amount of a transcript comprises cells.

19. The method of any one of aspects 15-18, wherein the one or more samples comprises blood buffy coat.

20. The method of any one of aspects 15-19, wherein the cells comprise white blood cells.

21. The method of any one of aspects 1-20, wherein the one or more samples used for detecting and/or quantifying an amount of a protein comprises blood or a component thereof.

22. The method of any one of aspects 1-21, wherein the one or more samples used for detecting and/or quantifying an amount of a protein comprises blood plasma.

What is claimed is:

1. A method of assessing fertility fitness in a female mammal, the method comprising:
in one or more samples collected from the female mammal
(a) detecting one or more single nucleotide polymorphisms (SNPs) set forth in
(i) Supplementary Information 3;
(ii) BovineHD500034888, BovineHD500034891, BovineHD500034893, BovineHD500034894, BovineHD500034895, BovineHD500034896, BovineHD500034897, BovineHD500034898, BovineHD500034899, BovineHD1200026258, BovineHD1200026258, BovineHD2700000503, BovineHD2500009748, BovineHD2600007565, BovineHD2600007566, BovineHD1600016722, BovineHD1300006153, BovineHD2300004608, BovineHD0500034892, BovineHD0300007001, BovineHD1200026258, BovineHD2700000503, BovineHD1600016722, BovineHD0500034794, BovineHD0100008466, BovineHD2300004608, BovineHD2100009580, BovineHD2100009629, BovineHD2600011917, BovineHD0500035192, BovineHD0500035196; and/or
(iii) BovineHD500034888, BovineHD500034891, BovineHD500034893, BovineHD500034894, BovineHD500034895, BovineHD500034896, BovineHD500034897, BovineHD500034898, BovineHD500034899, BovineHD1200026258;
(b) determining an amount of a transcript of one or more genes set forth in
(iv) Supplementary Information 4;
(v) ENSBTAG00000021346, ENSBTAG00000047139, ENSBTAG0000003279, ENSBTAG0000015041, ENSBTAG0000007389, ENSBTAG0000000810, ENSBTAG0000040199, ENSBTAG0000002972, ENSBTAG0000004278, ENSBTAG0000019300, ENSBTAG00000012263, ENSBTAG00000018339, ENSBTAG00000007566, ENSBTAG00000015061, ENSBTAG00000054926, ENSBTAG00000018655, ENSBTAG00000021346, ENSBTAG00000040065, ENSBTAG00000009479, ENSBTAG0000005211, ENSBTAG00000018339, ENSBTAG00000054926, ENSBTAG00000040065, ENSBTAG00000009479, ENSBTAG00000047139, ENSBTAG00000003279, ENSBTAG00000015041, ENSBTAG00000007398, ENSBTAG00000000810, ENSBTAG00000040199;
(vi) ENSBTAG00000012263, ENSBTAG00000018339, ENSBTAG00000007566, ENSBTAG00000015061, ENSBTAG00000054926, ENSBTAG00000018655, ENSBTAG00000021346, ENSBTAG00000040065, ENSBTAG00000009479, ENSBTAG0000005211; and/or from
(vii) one or more genes selected from the group consisting of ArfGAP with SH3 domain, ankyrin repeat and PH domain 3 (ASAP3), ATP synthase membrane subunit c locus 1 (ATP5MC1), Centrosomal protein 170 (CEP170), Myeloid derived growth factor (MYDGF), Coiled-coil domain containing 34 (CCDC34), RAD51 associated protein 1 (RAD51AP1), and Ubiquinol-cytochrome c reductase complex III subunit VII (UQCRQ);
(c) determining an amount of one or more proteins set forth in
(viii) Table 2;
(ix) P00744; P02768, Q2HJF0, Q95121, A5D798, F1MZ96, Q2KIX7, G1K122, F6QND5, Q5E9E3, P19034, P02768, F1N102, F1MZ96, G3X6N3, Q2KIX7, G5E5V0, G3MY71, G1K122, Q5EA67, P19034, F1MYX5, P00744, AOA6B9SCM2, P02768, Q2HJF0, F1N102, Q95121, P80109, A5D798;
(x) P19034, F1MYX5, P00744, AOA6B9SCM2, P02768, Q2HJF0, F1N102, Q95121, P80109, A5D798; and/or
(xi) from one or more proteins selected from Apolipoprotein C-II (APOC2), Lymphocyte cytosolic protein 1 (LCP1), Vitamin K-dependent protein Z (PROZ), Albumin (ALB), Serotransferrin-like (LOC525947), Complement component C8 beta chain (C8B), Pigment epithelium-derived factor (SERPINF1), Phosphatidylinositol-glycan-specific phospholipase D (GPLD1), Alpha-ketoglutarate-dependent dioxygenase FTO (FTO); or
(e) from one or more metabolites, optionally wherein the metabolite is 2-Dehydro-D-gluconate; or
(d) any combination of (a)-(d).

2. A method of assessing fertility fitness in a female mammal, the method comprising:
in one or more samples collected from the female mammal
(a) detecting the presence one or more single nucleotide polymorphisms (SNPs) selected from rs110918927, chr12: 85648422 and rs109366560, chr11:37666527;
(b) detecting and/or quantifying an amount of a transcript of adipocyte plasma membrane associated protein (APMAP), dynein axonemal intermediate chain 7 (DNAI7), or both;
(c) detecting and/or quantifying an amount of Alpha-ketoglutarate-dependent dioxygenase FTO (FTO);
(d) detecting and/or quantifying an amount of 2-Dehydro-D-gluconate; or
(e) any combination of (a)-(e).

3. The method of claim 1, whereby detecting and/or quantifying (a), (b), (c), (d) or any combination thereof determines that the female mammal is fertile or subfertile.

4. The method of claim 1, wherein the female mammal is fertile when the female mammal is (a) homozygous for allele A at rs110918927, chr12: 85648422, (b) is homozygous for allele G at rs109366560, chr11:37666527 (c) has a greater amount of transcript from APMAP as compared to a suitable control, (d) has a lesser amount of transcript of DNAI7 than a suitable control, (e) has a greater amount of FTO, (f) has a greater amount of 2-Dehydro-D-gluconate than a suitable control; or (g) any combination of (a)-(f).

5. The method of claim 1, wherein the female mammal is subfertile when the female mammal (a) has at least one G allele at rs110918927, chr12: 85648422, (b) is homozygous for allele G at rs109366560, chr11:37666527, (c) has a lesser amount of transcript from APMAP as compared to a suitable control, (d) has a greater amount of transcript of DNAI7 than a suitable control, (e) has a lesser amount of FTO as compared to a suitable control, (f) has a greater amount of 2-Dehydro-D-gluconate than a suitable control; or (g) any combination of (a)-(f).

6. The method of claim 5, wherein the female mammal is subfertile when the female mammal (a) has one A allele and one G allele or is homozygous for the G allele at rs110918927, chr12: 85648422, (b) is homozygous for allele G at rs109366560, chr11:37666527, (c) has a lesser amount of transcript from APMAP as compared to a suitable control, (d) has a greater amount of transcript of DNAI7 than a suitable control, (e) has a lesser amount of FTO as compared to a suitable control, (f) has a greater amount of 2-Dehydro-D-gluconate than a suitable control; or (g) any combination of (a)-(f).

7. The method of claim 1, wherein the femail mammal is subfertile when the female mammal has more 2-Dehydro-D-gluconate than a suitable control, optionally wherein the suitable control is a fertile female mammal.

8. The method of claim 1, wherein the female mammal is a non-human animal or a human.

9. The method of claim 1, wherein the female mammal is a bovine, equine, ovine, porcine, canine, or feline.

10. The method of claim 1, wherein the female mammal is pre-pubertal, is pubertal, or is sexually mature.

11. The method of claim 1, wherein the one or more samples comprise a bodily fluid, optionally, wherein the one or more samples comprises blood or component thereof.

12. The method of claim 1, wherein.

13. The method of claim 1, wherein the one or more samples comprises plasma, buffy coat, or both.

14. The method of claim 1, wherein the one or more samples comprises cells.

15. The method of claim 14, wherein the cells are white blood cells.

16. The method of claim 1, wherein the one or more samples used for detecting one or more single nucleotide polymorphisms (SNPs), transcripts, proteins, or any combination thereof comprises cells.

17. The method of claim 15, wherein the one or more samples comprises blood buffy coat.

18. The method of claim 15, wherein the cells comprise white blood cells.

19. The method of claim 1, wherein the one or more samples used for detecting and/or quantifying an amount of a protein comprises blood or a component thereof.

20. The method of claim 1, wherein the one or more samples used for detecting and/or quantifying an amount of a protein comprises blood plasma.
